(19) United States
(12) Patent Application Publication
(10) Pub. No.: US 2017/0189640 A1
(43) Pub. Date: Jul. 6, 2017

(54) Circadian Rhythm Alignment Lighting

(71) Applicant: INNOSYS, INC., Salt Lake City, UT (US)

(72) Inventor: Laurence P. Sadwick, Salt Lake City, UT (US)

(21) Appl. No.: 15/321,174

(22) PCT Filed: Jun. 25, 2015
(86) PCT No.: PCT/US15/37838

§ 371 Date: Dec. 21, 2016

Related U.S. Application Data
(60) Provisional application No. 62/017,162, filed on Jun. 25, 2014.

(51) Int. Cl. A61M 21/02; H05B 33/08; H05B 37/02

(52) U.S. Cl. CPC A61M 21/02; H05B 37/0281; H05B 33/0896; H05B 33/0872; H05B 37/0272; A61M 2021/0044

(57) ABSTRACT

A lighting system includes a number of light sources with multiple light colors configured to affect circadian rhythms, and a power supply configured to power the light sources and to control an overall output color.

### BACKGROUND

Circadian rhythms are biological processes also referred to as an internal body clock that are important in governing sleeping patterns and normally oscillates with a period of about a day. Such rhythms are widely observed in humans, animals, plants, etc. Although circadian rhythms are endogenous or self-sustained, they are adjusted to the local environment by external cues including light and temperature. The biological processes referred to generally as circadian rhythms can include, for example, patterns of brain wave activity, hormone production, cell regeneration, and other biological activities. Circadian rhythms can be disrupted by a number of factors, including but not limited to shift work, pregnancy, time zone changes, medications, changes in routine such as staying up late or sleeping in, medical problems including Alzheimer's or Parkinson disease, mental health problems, etc., and can cause circadian rhythm disorders such as Jet Lag or Rapid Time Zone Change Syndrome, Shift Work Sleep Disorder, Delayed Sleep Phase Syndrome (DSPS), Advanced Sleep Phase Syndrome (ASPD), Non 24-Hour Sleep Wake Disorder, etc.

### SUMMARY

Various embodiments of the present invention provide lighting systems that can be used for circadian rhythm alignment.

The embodiments shown and discussed are intended to be examples of the present invention and in no way or form should these examples be viewed as being limiting of and for the present invention.

This summary provides only a general outline of some embodiments of the invention. The phrases “in one embodiment,” “according to one embodiment,” “in various embodiments”, “in one or more embodiments”, “in particular embodiments” and the like generally mean the particular feature, structure, or characteristic following the phrase is included in at least one embodiment of the present invention, and may be included in more than one embodiment of the present invention. Importantly, such phrases do not necessarily refer to the same embodiment. Additional embodiments are disclosed in the following detailed description, the appended claims and the accompanying drawings.

### BRIEF DESCRIPTION OF THE FIGURES

A further understanding of the various embodiments of the present invention may be realized by reference to the Figures which are described in remaining portions of the specification. In the Figures, like reference numerals may be used throughout several drawings to refer to similar components.

FIGS. 1A-1B depict front and back sides of a solid state lighting panel for use in a circadian rhythm alignment lighting system in accordance with some embodiments of the invention;

FIGS. 2A-2B depict front and back sides of another solid state lighting panel for use in a circadian rhythm alignment lighting system in accordance with some embodiments of the invention;

FIGS. 3A-3B depict front and back sides of another solid state lighting panel for use in a circadian rhythm alignment lighting system in accordance with some embodiments of the invention;

FIGS. 4A-4B depict front and back sides of another solid state lighting panel for use in a circadian rhythm alignment lighting system in accordance with some embodiments of the invention;

FIG. 5 depicts a solid state fluorescent lamp replacement for use in a circadian rhythm alignment lighting system in accordance with some embodiments of the invention;

FIG. 6 depicts an example power supply circuit in a solid state fluorescent lamp replacement for use in a circadian rhythm alignment lighting system in accordance with some embodiments of the invention;

FIG. 7 depicts an example power supply circuit in a multiple-ballast solid state fluorescent lamp replacement for use in a circadian rhythm alignment lighting system in accordance with some embodiments of the invention;

FIG. 8 depicts a control circuit that can be used to set the output current or voltage of a solid state fluorescent lamp replacement for use in a circadian rhythm alignment lighting system in accordance with some embodiments of the invention;

FIG. 9 depicts an example rectifier that can be used in a solid state fluorescent lamp replacement in a circadian rhythm alignment lighting system in accordance with some embodiments of the invention;

FIG. 10 depicts an example rectifier that can be used in a solid state fluorescent lamp replacement in a circadian rhythm alignment lighting system in accordance with some embodiments of the invention;

FIG. 11 depicts an example rectifier that can be used in a solid state fluorescent lamp replacement in a circadian rhythm alignment lighting system in accordance with some embodiments of the invention;

FIG. 12 depicts an example rectifier that can be used in a solid state fluorescent lamp replacement in a circadian rhythm alignment lighting system in accordance with some embodiments of the invention;

FIG. 13 depicts a protection mode circuit that can be used in a solid state fluorescent lamp replacement in a circadian rhythm alignment lighting system in accordance with some embodiments of the invention;

FIGS. 14-19 depicts various configurations of multi-color solid state lights that can be used in a circadian rhythm alignment lighting system in accordance with some embodiments of the invention; and

FIGS. 20-22 depict an example power supply circuit with various input connections in a multiple-ballast solid state fluorescent lamp replacement for use in a circadian rhythm alignment lighting system in accordance with some embodiments of the invention.

### DETAILED DESCRIPTION OF THE INVENTION

The present invention relates to solid state lighting systems that permit enhanced circadian rhythm alignment and maintenance using light. Such sources of light include, but are not limited to, computer screens, monitors, panels, etc., tablet screens, smart phone screens, etc., televisions (TVs), LCD and CRT displays of any type or form, DVD and other entertainment lighting and displays containing LEDs, OLEDs, CCFLs, FLs, CRTs, etc., displays, monitors, TVs, OLED, LED, CCFL, FL, incandescent lighting, etc.

The present invention can use smart phones, tablets, computers, dedicated remote controls, to provide lighting appropriate for circadian rhythm alignment, correction, support, maintenance, etc. that can be, for example, coordinated wake-up and sleep times whether on a ‘natural’ or shifted (i.e., night workers, shift workers, etc.) to set and align their sleep patterns and circadian rhythm to appropriate phases including time shifts and time zone shifts due to work and other related matters.

The present invention can use external and internal information gathered from a number of sources including clocks, internal and external lighting, time of the year, individual, specific input, physiological signals, movements, monitoring of physiological signals, stimuli, including but not limited to, EEG, melatonin levels, urine, wearable device information, sleep information, temperature, body temperature, weather conditions, etc., combinations of these, light levels including sunlight levels, etc.

The present invention can, for example, use shorter (i.e., blue) wavelength light to stimulate and awaken or support waking and healthy state functionality and use longer (i.e., yellow, amber, red, etc.) wavelength light to promote sleep and rest state. For example, amber light emitting diodes (LEDs) and/or organic light emitting diodes (OLEDs) can be used for sleep and blue LED(s) or OLED(s) or other sources of light including but not limited to quantum dots (QDs) for waking and to simulate the exposure to natural sunlight. Other colors including but not limited to orange, yellow-orange, yellow, etc. can also be used. The term color as it is used herein can refer to a single wavelength of light or range of wavelengths of light, to multiple wavelengths that are visually blended. The LEDs, OLEDs, QDs, etc. can be separate colors, panels, or integrated, layered, etc. colors on the same panel and can be of any type and construction. Embodiments of the present invention can use external information such as time of day/night, light levels, computers, websites, smart phones, clocks, atomic clocks and other wired and wireless timing information including weather and weather-related information, time of sunrise and/or time of sunset, etc. light levels including but not limited to sunlight levels, etc., combinations of these, etc., to determine whether to have amber (or yellow or red, etc.), blue or both turned on. AC power, solar power, batteries, or a combinations of these, etc. can be used to provide power to the OLEDs, LEDs, QDs, other types of SSL, combinations of these, etc. Embodiments of the present invention can use a portable LED, OLED, QD, combinations of these, etc. panel or panels, other types and sizes (from small to very larger and bigger including tiled, stacked, etc.) panels including troffers, task lamps, bed lamps, table lamps, under counter, over counter, vanity, wall, ceiling, sconce, luminaries, sleep detectors, wearable sleep detectors and circadian rhythm detectors, etc. Embodiments of the present invention can be a fluorescent tube replacement of any length and any diameter that contains multiple color light sources with or without a white light source or with more than one white light source including white light sources having different color temperatures, which can be controlled (i.e., turned on, dimmed), for example, but not limited to, in ways to produce shorter visible wavelength containing light for waking up and waking hours and produce longer visible wavelength containing light with the absence of or greatly reduced shorter wavelength content light for sleeping and resting as well as other types of lights including but not limited to A lamps (including E26 and E27 socket lamps), PAR lamps (including PAR30 and PAR38), R lamps (including R30, R40), flood lamps, PL 2 or 4 pin lamps, MR lamps (including MR16), GU lamps (including GU10), T12, T10, T9, T8, T5, T4, etc., 1 ft, 2 ft, 3 ft, 4 ft, 5 ft, 6 ft, 8 ft, less than 1 ft, greater than 4 ft, greater than 8 ft, 18 inches, etc., high intensity discharge lamps (HID) of any type and form, 2×2 ft, 4×4 ft, 2×4 ft, 4×2 ft, 1×1 ft, etc. low voltage lamps, low voltage magnetic lighting, etc., virtually any type of light form factor light source, combinations of these, etc. Embodiments of the present invention can include circuit implementations that are able to receive and ‘read’, for example, ‘atomic clock’ signals that can be used with other information about geographic location. Such time and position information can, for example, be obtained automatically by using, as an example, a global positioning system (GPS)—which also have their own atomic clocks—which can receive the 60 kHz low frequency transmission, for example sent/transmitted in the USA from Colorado—and the same frequency or relatively similar frequencies in other countries and continents. Such time and position information can be used to set the Circadian Rhythm system to the ‘proper’ phase. In some embodiments of the present invention, the ‘proper’ phase can be overridden and set to a different part of the phase, for example, for shift workers who work at night and sleep during the day or part of the day. This could be manually or automatically determined and set based on, for example, the work and sleep schedule of an individual or groups of individuals, along with potentially other information, etc.

Blue OLED(s) and/or LEDs can be used in light therapy or circadian rhythm treatments to be controlled (i.e., turned on, dimmed) based on weather and/or ambient light conditions, for example based on weather reports in overcast, stormy, gloomy, rainy, winter or otherwise dismal weather. The weather or other conditions can also be determined by sensors such as, but not limited to, light, solar, humidity, temperatures, moisture, spectral and/or precipitation sensors, in some cases in combination with weather reports from one or more sources. Such embodiments of the present invention can also be used for other types of light therapy including treating seasonal affective disorder (SAD) and other types of health issues including but not limited to Alzheimer's, Parkinson disease, mental health problems, Jet Lag or Rapid Time Zone Change Syndrome, Shift Work Sleep Disorder, Delayed Sleep Phase Syndrome (DSPS), Advanced Sleep Phase Syndrome (ASPD), Non 24-Hour Sleep Wake Disorder, etc., combinations of these, etc.

The present invention can use televisions (TVs) essentially of any type or form, including, but not limited to smart TVs, NTSC, PAL, flat screen, etc. TVs and related and similar items, products and technologies including, but not limited to, computer and other monitors and displays that can either be remotely or manually controlled in any way or fashion and, in some embodiments, monitored. The present invention can use smart phones, tablets, PCs, remote controls including programmable remote controls, consoles, etc., combinations of these etc., to control and set the content of the lighting (e.g., white or blue-enriched, etc. combinations of these, etc. for wake-up; yellow, amber, orange, red, etc., combinations of these, etc. for sleep-time, etc.) automatically to assist in circadian rhythm, sleep, SAD mitigation, reduction, elimination, etc. In some embodiments of the present invention, music, sounds, white noise, sea shore sounds, sound effects, narratives, live audio, inspirational audio including previously recorded, generated, synthesized, etc., soothing sounds, familiar sounds and voices, etc. and combinations of these to go to sleep with. Jarring, buzzing, alarming, beeping, interrupting sounds, alarm clock sounds and noises, sleep disruptive sounds, noises and/or voices, etc. accompanied by white light, blue color/wavelength light including, but not limited to, slowing dimming up to a preset, optimum, and/or maximum brightness or setting, etc. are implemented in some embodiments for wake-up in the morning. Embodiments of the present invention can provide multiple wakeups to the same location and/or different locations including other locations in homes, houses, hotels, hospitals, dormitories including school and military and other types of barracks, dormitories, etc., assisted living homes and facilities, chronic care facilities, rehabilitation facilities, etc., children's hospitals and care facilities, etc. group living, elder living, assisted living, etc., children's rooms and other family members whether in the same physical location or in different physical locations, friends and family, clients, guests, travelers, jet lagged and sleep deprived people and personnel, etc. Embodiments of the present invention can also be turned on at full (maximum) brightness, for example but not limited to, at wake up with as bright and intense of a light as possible, etc. Thus, some embodiments enable the scheduled programming of intensity as well as color and/or wavelength, based on time of day or other temporal and/or location and/or other ambient condition information or based on any other information, etc.

The present invention can also be used to assist in mitigating or alleviating seasonal affective disorder (SAD) as well as supporting circadian rhythm and promote healthy sleep. Embodiments of the present invention can be fully dimmed and are fully dimmable both remotely and locally/manually. For example, for SAD therapy, the light or lights could be turned on to maximum brightness including white and/or blue brightness/intensity for a selected period of time which could be manually, automatically, programmed, preprogrammed, scheduled, sequenced, as a wake up or part of a wakeup, etc. In some embodiments of the present invention, the light intensity may be monitored and recorded as well as monitoring and recording other aspects and parameters including human and or animal health information, state, vital and other signs, etc.

Example embodiments of the present invention allow for friends and family and others (as so allowed) to be able to program in wake-up and sleep information for an individual or groups of individuals to be remotely synched up for circadian rhythm phases including wake up (e.g., bright white and/or blue wavelength/color and similar such colors and wavelengths along with appropriate sounds, noises, beeps, buzzing, bells and whistles, etc. coupled with or in place of personal prerecorded or live words from the initiating party or person(s), etc. At sleep time, the lights would switch to wavelengths below those that, for example, suppress melatonin generation, etc. such as, but not limited to, yellow, orange, amber, red, etc. which could be accompanied by soothing and related sounds, noises, voices, etc. including but not limited to inspirational, spiritual, personalized, melodies, poems, words including custom pre-recorded or live voices, etc.

For example, the present invention can be implemented so that the user can configure and set the hardware and software interface of the circadian rhythm cycle lighting system and/or, for example, the color-changing including white color changing lighting system so as to, for example, but not limited to, individually input, control, program, interact with, monitor, log, etc. the circadian rhythm lighting system. Embodiments of the present invention can include motion detection/proximity detection/RF detection and decide/determine which color(s) of light to produce, in conjunction and coupled with other sensors, detectors, counters, timers, clocks, etc., including for example but not limited to, sound, photo, light, spectrum, voice, detectors and sensors to turn on to maintain the appropriate circadian rhythm cycle regulation, etc. Motion sensors can perform multiple duties—turning on/off lights, alerting that occupants, intruders, others, etc. are present, heating or cooling spaces, burglar alarm, etc. For example, implementations can turn on and set the hall and other lights to blue enhanced light in, for example, the morning, day or afternoon phases of the circadian rhythm cycle and turn on and set the hall or other lights to blue depressed or blue eliminated light in, for example, the evening, night or night time/sleep time phases of the circadian rhythm cycle. In addition the lights/lighting can be dimmed at any point in the cycle that is appropriate or needed especially at nighttime including, for example, but not limited to, both automatically and manually. For example, implementations of the present invention can turn on and set the kitchen lights to blue enhanced light at, for example, breakfast or lunch and possibly dinner and turn on and set the hall or other lights to blue depressed or blue eliminated light (i.e., red, amber, orange, yellow, etc.) in, for example, possibly at dinner or for after dinner snacking, etc. Other situations can include, for example, turn on and set the bedroom lights to blue enhanced light in, for example, the morning, day or afternoon phases of the circadian rhythm cycle and turn on and set the hall or other lights to blue depressed or blue eliminated light in, for example, the evening, night or night time/sleep time phases of the circadian rhythm cycle. For example, embodiments of the present invention can turn on and set the bathroom lights to blue enhanced light in, for example, the morning, day or afternoon phases of the circadian rhythm cycle and turn on and set the hall lights to blue depressed or blue eliminated light in, for example, the evening, night or night time/sleep time phases of the circadian rhythm cycle. Embodiments of the present invention can use red, green, blue, amber, white LEDs, OLEDs, QDs, other colors of LEDs, OLEDs, QDs and white LEDs, OLEDs, QDs, etc., subsets and combinations of these, etc. Embodiments of the present invention can use RGB OLEDs and LEDs and/or QDs and combinations of RGB OLEDs, LEDs, QDs and white LEDs, OLEDs, QDs, etc. for the lighting. Blue light in general can also include one or more white lights having one or more color temperatures (i.e., but not limited to 2700 k, 3000 k, 4000 k, 5000 k, 6000 k, etc., cool white, warm white, bright white).

The present invention can also provide two or more side (multi-side) lighting for example, for a fluorescent light replacement (FLR) where one side contains a solid state light (SSL) that, for example, consists of white color or white colors of one or more color temperatures and another side contains SSL or other lighting of one or more wavelengths such as red, green, blue, amber, white, yellow, etc., combinations of these, subsets of these, etc. The two or more sided lighting can perform different functions—for example, the side that is primarily white or all white light of one or more color temperatures can provide primary lighting whereas the side that has one or more color/wavelengths of light can provide indication of location, status, code level in, for example, a hospital (i.e., code red, code blue, code yellow, etc.), accent lighting, mood lighting, location indication, emergency information and direction, full spectrum lighting, etc. Some embodiments of the present invention can use multi-SSL packages, for example, multi-LED packages that have more than one LED on a package; as an example, a multi-LED package that contains one or more white color temperatures having different kelvin ratings, an amber LED and a blue LED. Such a package can provide different white combinations along with enhanced blue wavelength content to support wake up for circadian rhythm support as well as amber color to support falling asleep and sleep and also for short wake-up periods to get up to, for example, go to the bathroom and then go back to sleep. In addition to the multi-white color with blue and amber, other colors can be included or substituted including, but not limited to yellow, green, red, orange, other whites, additional whites, purple, yellow-orange, etc., combinations of these, more than one of these, etc.

The present invention can be used to aid in circadian rhythm regulation and cycle synchronization as well as Seasonal Affective Disorder (SAD). The present invention can aid in correcting sleep disorders and provide light therapy including for SAD. The present invention can use input, feedback, etc. including human physiological and biological input and feedback and environmental (including, but not limited to, temperature, time of day or night, ambient light, light spectrum, etc.) to control and monitor the light including the colors/wavelengths and/or the intensity of the light, etc. as well as have the light and the effects of the light including biological and/or physiological monitored, analyzed, etc. as well.

The present invention can be used for personal or professional use and applications. The present invention can be used, for example, in hospitals, rest homes, senior care homes, rehabilitation facilities, short term and long term care facilities, hospitals, libraries, homes, residences, commercial and industrial buildings and locations, schools including K12, universities, colleges, etc., in cleanrooms, in confined spaces, in spaces devoid of natural light, on ships, buses, boats, planes, aircraft, submarines, vessels, all times of marine, ground, air and space vehicles including transport and working environments, spaces, vehicles, etc.

The present invention can use actimetry, sleep actigraphs which can be of any form including watch-shaped and worn on the wrist of the non-dominant arm, temperature, EEG, wrist, body movements, polysomnography (PSG) and other such techniques, etc.

The present invention can also be used to provide relatively dim illumination at night of appropriate wavelengths and can be integrated into a single light source and sensor unit to provide lighting sufficient for sleeptime/nighttime use and egress for, for example, children and adults including more aged and senior adults and parental or other (including, but not limited to nursing, nurse assistant, care giver, hospital, rest home, hospice, trauma, emergency room and similar environments, recovery, rehabilitation, assisted living, elderly living, senior care, etc. centers/facilities, etc.), support staff, cleaning personnel, etc., dementia of all types and forms, etc., and to provide various types of light therapy including but not limited to individual, customized, programmable, adjustable, adaptable, sequenced, setting, synchronized, etc. The present invention lighting can be used for, for example but not limited to, seniors, families, businesses, residences, homes, houses, elderly, physically impaired people and persons, etc. to signal, alarm and/or alert others of an emergency, an intrusion, a fire, a fall, an injury, toxic or explosive gases, loss of heating, water leakage, too much noise, too little noise, too much talking, too little talking, no talking, no noise, noise, sound, talking, etc. in one or more frequency ranges, etc., by for example flashing lights, on-off lights at certain periods of repetition, different colors flashing, different patterns of colors, different intensities and dimming, etc., combinations of these, etc. In some cases, the interior/indoor lights can be set to full on/full brightness while the exterior/outdoor lights can be set to flashing or other modes including but not limited to those discussed herein. In some embodiments audio alarms including but not limited to sirens, recorded or synthesized voice messages, actual sounds from microphones within the house, synthesized ring tones, alarms, alerts, etc., other types of patterns of sound, music, etc., combinations of these, etc. can be used.

The present invention can be used in libraries, classrooms, offices, hospitals, quiet zones, private property, doctor's offices, work places, etc. to provide responses to too much noise, too much talking, too much movement, too little movement, intrusion into certain areas, etc. by, for example, changing light intensity, color, turning on or off, flashing, etc. As an example one or more people may be talking and should the level of talk reach a certain level, one or more of the lighting may change to red or remain white with some red included or flashing until the level of the talk decrease to a certain value, or the light may be dimmed and/or the color changed temporarily in either a constant or flashing fashion, or may be changed (dimmed, etc.) until sound levels or other stimuli have returned to conditions associated with normal operation, for example until sound levels have dropped below a threshold.

The present device can be made into light sources, including but not limited to sheet light sources, which can incorporate solar cells either on the front or the back, and optional energy storage such as batteries to create a light source that can be powered when there is no sunlight or can also act as a privacy screen and/or temperature reducer over windows by absorbing and blocking the sunlight (and potentially associated heat and UV rays) from entering the space on the interior side of the window while still powering and providing energy to the light sources to illuminate the interior space(s).

The present invention can use projectors, television sets, computer monitors and other displays, etc. including all or some of these, etc. as light sources and to provide light of various and different colors including different white light colors including for use in light therapy including but not limited to circadian rhythm. SAD, dementia, other maladies, illnesses, diseases, etc., combinations of these, etc. Implementations of the present invention can include using televisions including older televisions that can be switched on and set to appropriate wavelengths for waking up and appropriate wavelengths for resting/going to sleep, etc. Embodiments of the present invention can use an interface/conversion/communication device/box/unit/etc. that can, for example, use the duplication of the remote control signals to turn on the television and set the channel such that the signals applied to the specifically set channel produce the desired wavelength spectrum. Embodiments of the present invention can also use a remotely controlled switch to turn on the television, projector, etc. Other video information and signals can be used including computer, web, cable, satellite, cellular phone, etc. video streaming or any type or form, etc., combinations of these, etc. Audio signals may also be used and applied to assist in waking or sleeping, such as, but not limited to, synthesized, simulated, emulated, and/or recorded voices, sounds, environments, tones, natural or man-made sounds, live streaming, personal communications, television, radio, other broadcasting whether wireless, web-based, cable, satellite, wired, etc., combinations of these, alarm clocks, either alone or in combination with changing light levels and/or wavelengths, in order to provide predetermined, or programmable, randomized, live, etc., audible and/or light-based alarms, whether gradual, gentle, insistent, etc. Such alarms can be adapted for slow or fast waking of individuals with a range of light sleeper to deep sleeper characteristics. Changing light patterns in alarms can simulate sunrise or other conditions, etc. or in certain cases, sunset or other times of the day or night, etc. which can be customized and personalized for a person, persons, groups of people, etc.

Embodiments of the present invention can also measure the input and output power of individual and/or groups of light source, one or more light sources, etc. including but not limited to input current, input voltage, input power, input real power, input VA, power factor, total harmonic distortion (THD), individual harmonics, in rush current, frequency, one or more temperature, etc., output current, output voltage, output power, output real power, output VA, output power factor, output total harmonic distortion (THD), individual harmonics, output in rush current, one or more temperature, etc., efficiency, input/output efficiency, etc., and keep track of the status, health, operating conditions, etc. analytics of one or more or all of the lighting as well as other electricity consuming and generating sources, etc. (← Guy, more help here). In some embodiments of the present invention, the health and status including but not limited to faults, alerts, poor performance, need of repair or maintenance, etc., of the lamp can be transmitted to one or more locations including but not limited to cell phones, smart phones, tablets, computers, servers, etc. including information that can be used for analytics, maintenance, abnormal behavior or operation, detection of movement in embodiments equipped with motion detection/proximity detection, etc., cell phone traffic, Bluetooth traffic, etc.

The present invention can be used to gently or urgently or anything in between wake a person or people by providing light with high/significant or total blue wavelength content. Such implementations of the present invention can be used in one or more locations that are collocated/local or located miles or continents apart. The present invention can control and monitor one or multiple light sources in one or more locations. For example parents can set one or more wake up sequences where the light can, for example, but not limited to, dim up slowly or go to full brightness instantly, provide vocals including, but not limited to, music, horns, buzzer, alarm, synthesized sounds, noise, nature, ocean and other sounds, combinations of sounds, voices, familiar voices, voice generated or previous voice recorded, etc. In a similar fashion, the present invention can include night-time or sleep time to control and monitor one or more light sources and optionally electrical outlets such as, for example, but not limited to, to control the turn off, dimming including gradual or abrupt or anything in between the light sources in one or more locations including the same or different rooms which could be set to simultaneously, separately, staggered, or other scheduling or sequencing of the light and related control. In some embodiments of the present invention, the amplitude of a sound, noise, acoustic, thud, vibration, mechanical, sounds associated with movement can be detected and optionally amplified including remotely amplified using commands, automatic signals, remote control and signals, etc. Embodiments of the present invention can also detect motion, noise, movement, voice, speech, exit or entrance in sleep including REM sleep, as well as other types and forms of acknowledgements etc. which, can for example, be used to determine if a person or persons responded,

Embodiments of the present invention can also use an infrared to RF wireless universal interpreter/converter as described in PCT Patent Application PCT/US15/12965 filed Jan. 26, 2015 for “Solid State Lighting Systems” which is incorporated herein by reference for all purposes. Such a universal interpreter/converter allows control of portable devices such as portable air conditioners, window air conditioners, portable heaters and furnaces, portable space heaters, portable space coolers, etc., entertainment devices, units, systems, etc., humidifiers, etc. In some embodiments of the present invention the infrared to RF wireless universal interpreter/universal converter/adapter may be installed in and included as part of a lamp, bulb, light fixture, etc., may be battery operated with a solar charger, a mechanical energy charger, other types of energy harvesting, etc. Such implementations of the present invention can use one or more mobile, portable wireless devices including, but not limited to, remote temperature sensors, smart phone temperature sensors and measurement devices, integrated circuits, etc., Bluetooth temperature sensors and measurement devices, tablet temperature sensors, etc., humidity sensors and measurement devices, etc. One or more of these sensors in one or more nearby locations may be used, for example, as temperature control points/locations for which certain embodiments of the present invention can be commanded to modify the temperature until one or more of the temperature setpoints are reached and maintained. Some embodiments of the present invention can also monitor the power (i.e., voltage, current, apparent power, real power, power factor, etc.) to monitor, store, calculate, make decisions, provide analytics, etc. of the heating and cooling energy use, etc.

The present invention may be used as a light source for multiple purposes including as a reading lamp, as a task lamp, as an ambient lamp, as a circadian rhythm regulator and adjuster, etc., an entertainment and mood lamp, emergency indicator or other indicator, guide light by shining or flashing different colors to indicate one or more paths simultaneously, sequencing including temporally sequencing the lighting to indicate directions to follow/take/etc., turning different parts including light source parts to indicate a direction or path, etc. to follow, a status indicator by shining various colors in various locations according to conditions to be identified, etc. Such emergency or identification or guide or other functions can be performed in combination or conjunction with other functions, including simultaneous lighting such as combining white illumination with colored indicators.

An example of the present invention includes, but is not limited to, a light source for train, bus, airplane, ship, boat, yacht, recreational vehicle (RV), SUV, limousine, van, submersible vehicles including, but not limited to, submarines, Navy boats, commercial jets, plant growth, etc.

The present invention can be used to produce various effects in, for example, a long distance travel by train, boat or plane in which the users can choose from soothing or exciting colors, certain wavelengths of light to help induce, reset, etc. circadian rhythms and melatonin production or suppression, etc., to address SAD conditions, to provide one or more types of light therapy, to provide a calming or exciting ambiance, to affect mood, emotions, sleep, rest, enjoyment, ambiance, environment, relaxation, alertness, focus, attention span, etc.

The present invention can be used, for example, on a commercial airplane to allow the passenger to adjust the local lighting by using, for example, voice, Bluetooth, WiFi, or any other wireless method, way, protocol, etc. to, for example, communicate with the light/lamp to dim, change color temperature, change color or combinations of colors to change white color temperatures, to provide alerts, alarms, mood setting, light therapy, turn off, turn on, tilt, and/or combinations of these, etc.

The present invention can be attached/embedded/incorporated/integrated/etc. into a fan, including, but not limited to, a ceiling fan that in some embodiments can change speed and light intensity and/or colors as it rotates. The LED and/or OLED and/or QD lighting can be incorporated/attached/embedded/etc. on one or both sides of the fan blades as well as other parts of the fan.

As an example of the present invention, a 12 channel driver can separately and independently supply and wired and/or wirelessly control (i.e., dim) each color of four RGB or three RGBA or RGBW SSL panels as well as 12 individual monochrome (e.g., white or other color) SSL panels, and/or a mix and match combination of both color, color-changing and/or white SSL panels or color changing and one or more white color temperatures. Of course more or less channels can be implemented.

The present invention can implement building block power supply approaches that can be mated with and sold with SSL panels, lightbars, lamps, strings, etc. as SSL lighting kits.

The driver electronics for the color changing/tunable SSL lighting allow, among other things, flexible, selectable lighting including warm, cool, daylight, etc., white light choices for residential consumers and business customers. These drivers also permit and support remote dimming, control, monitoring, data logging, light and driver electronics health as well as analytics.

All of the above can be wirelessly interfaced, controlled and monitored using, for example, smart phones (i.e., iPhones, Androids), tablets (i.e., iPad, iPod touch, Droid, Kindle, Samsung, Dell, Acer, Asus, etc. tablets), laptops, desktops and other such digital assistants and/or by wired or powerline communications.

The universal drivers can also support Triac and 0 to 10 Volt dimming as well as optional powerline (PLC) and wired and/or wireless remote control. Embodiments may either be AC or DC input power and the power supply/driver can support 0 to 10 volt dimming and can have optional wired and/or wireless control and monitoring.

Some embodiments of the present invention include power supplies and drivers specifically focused on OLEDs that address both the rather unique properties of OLEDs compared to, for example, even LEDs. In general, both OLEDs and LEDs should be current control driven—that is to safely operate both LEDs and OLEDs the power source should be current controlled and regulated as opposed to, for example, applying a constant, regulated voltage to the OLEDs or LEDs.

In general LEDs are point sources made up of certain mixtures/alloys of III-V semiconductors based, for example, binary gallium arsenide (GaAs) and gallium nitride (GaN) forming ternary alloys such as, but not limited to, aluminum gallium arsenide (AlGaAs) and aluminum gallium nitride (AlGaN). These and other such alloys allow a vast number of nearly single wavelength with a relatively small full width at half maximum (FWHM) optical emission which can include optical emission wavelengths that are visible to the human eye and are perceived as colors. White light LEDs can be achieved in a number of ways including color combining single color LEDs such as red, green and blue LEDs or using phosphors or QDs to perform wavelength conversion(s). LEDs are two terminal point source emitter devices which emit light when an electrical stimulus is applied. LEDs can be easily formed into parallel and/or series configurations occupying relatively small areas. OLEDs, on the other hand, are made of molecules that also emit light when electrical stimulus is applied. However, unlike LEDs, OLEDs are designed and configured as area sources and not point sources. There are a number of ways to also obtain white light OLEDs including homogenously mixing at, for example, the nanometer level red, green, blue or red, yellow, blue or other combinations of OLEDs, stacking layers of various colors of OLEDs vertically on top of each other, having stripes of various colors placed laterally close to each other, etc.

With LEDs, typically both the cathode and anode are available for, for example, each individual LED color to be connected in parallel and/or in series either individually or in groups/arrays/etc. such that often there are only two electrical power connections from the power to the LEDs and therefore the power supply/driver output and output connection configurations are often much simpler and more universal for LEDs than OLEDs. Of course, with the continued widespread growth and use of LEDs, there are and will be numerous exceptions to just the two connections per LED fixture or luminaire although such a generalization usually applies to LED lights and lamps such as, but not limited to, GU10, MR16, A Lamps, PAR 30, PAR 38, R30, T4, T5, T8, T9, T10, T12, PL 2 and 4 pin, and other SSL/LED/OLED/QD/etc. lamp replacements. Unless there is only one OLED panel that has only two electrode connections for a given lighting application, an optimized power supply design for multi-electrode (i.e., more than two electrodes) OLED panel(s) can involve consideration of a number of factors including, among others, ensured proper current sharing, size/gauge of wires used, over-current protection, over-voltage protection, individual OLED panel fault detection/correction, OLED lifetime aging, OLED differential color aging (e.g., blue color lifetime being lower than typically other OLED colors), whether to put multiple OLED panels in parallel or series or combinations of both, voltage drops in the interconnect wiring between the power supply and the OLED panels for OLED fixtures and luminaries.

The present invention provides solutions that include OLED lighting kits that would include power supplies/drivers, connectors/interconnects and OLED panels that are all designed to be mated to each other. In addition interfaces can provide significant assistance and aid in connecting multiple OLED panels to power supplies and drivers safely and correctly. This simple interface will use an OLED identification system that allows the power supply/driver and each of the individual OLED panels to communicate with each other in a similar but much simpler (and slower) fashion as, for example, the Telecommunications Industry Association/Electronic Industries Alliance (TIA/EIA) 485 also known as RS485 interface (which is also the basis of, for example, Modbus, Profibus, DMX512, etc.) 2 wire systems.

The OLED power supplies and example associated innovative lighting and luminaire applications including the circadian rhythm cycle regulation lighting system can also be portable OLED or LED lighting that can be charged by AC, direct current (DC) or solar power/energy sources. Such innovative OLED and LED lighting can be used for camping, emergency, outdoors, indoors, and general portable, etc. compact and rechargeable illumination applications including circadian rhythm regulation, SAD and other types of light therapy applications in these varied environments, etc. With properly designed high efficiency power supplies/drivers, portable OLED and LED lighting sources provide highly innovative, attractive, flexible and even colorful and also entertaining lighting as well as being lightweight and able to support novel shapes and form-factors while still providing circadian rhythm cycle regulation that can be individually modified and adjusted for these and other (e.g., work time, work space, shift time, etc.), environments.

The present invention includes OLED power supplies and associated OLED lighting for desk, and task applications and innovative color changeable OLED RGB (or RYB, RGBA, RTBA, RGBAW, RGBYW, etc. and/or additional colors, etc.) power supplies and drivers that can be produced cost-effectively with excellent performance, efficiency, efficacy, etc. The embodiments of the present invention are very flexible in design and application space.

The present invention includes power supplies for OLEDs, LEDs, QDs, etc. including ones designed for universal AC or DC input voltages and Triac and other dimming formats including 0 to 10 V, powerline, wireless, etc. Such power supplies can be adapted to be highly efficient—in some power supply/driver cases of close to 90%, even with relatively low output voltage (˜6.3) and relatively high current (close to 1 amp) for a ˜6.3 Watt OLED lamp output in an example embodiment. Embodiments of the present invention include a number of high performance power supplies and drivers for both monochromatic and multiple color/color changing/color tunable OLED lighting panels, including for example 12 channel common anode and/or common cathode OLED drivers that can be individually addressed and controlled/dimmed by wired and wireless interfaces and smart dimmable OLED desk/task lamps.

In an example embodiment of the present invention, portable wireless controlled lighting for the circadian rhythm regulation system can be set to white, blue (for wake-up), green, red, yellow (for blue-free light to promote sleep) and amber-orange (also for blue-free light to promote sleep).

To appropriately synchronize daily rhythms in behavior, physiology and brain functioning with environmental time, terrestrial species have evolved an endogenous, circadian timekeeping system. Circadian rhythms are generated by a hierarchy of central and peripheral oscillators with the suprachiasmatic nucleus (SCN) of the anterior hypothalamus acting as the master circadian pacemaker. The circadian system evolved such that environmental light input from the retina synchronizes internal timing, with the daily environmental cycle of sunlight and darkness as the primary time setter and keeper.

Artificial lighting has led to unnatural light overexposure, and persistent pattern changes have impacted circadian rhythms and sleep physiology. Numerous findings indicate that these changes have led to some degradation of mental and physical health among human populations. For example, flight attendants frequently traveling across time zones exhibit gross cognitive deficits associated with reductions in temporal lobe structures. Likewise, numerous studies indicate that circadian disruption leads to an increased incidence of cancer, diabetes, ulcers, hypertension and cardiovascular disease, and a degradation of mental health. Finally, it is clear that exposure to artificial light at night causes circadian rhythm misalignments leading to cognitive decline, increased incidence of depression and anxiety disorders, and a host of metabolic disorders. There are concerns regarding circadian rhythm misalignments as they are known to affect response time, judgment and planning, as well as psychomotor skills, and can increase the prevalence of certain illnesses and chronic issues.

By developing strategies to correct/mitigate disruptions to circadian function and misalignment between endogenous cycles in circadian and sleep physiology with the external environment (e.g., following jet lag, shift work, night work, etc.), one can recover diminished human performance as well as improve human health, reduce risk of disease, and enhance cognitive functioning and performance. For example, a wearable device can be used with a wireless system that can be utilized as a personal circadian rhythm monitor and regulation device capable of rapidly realigning the circadian rhythm of users to the local environment. In other situations the system adjusts the user to the work, mission or sleep cycle requirements, leading to improved sleep and performance. The lighting system continuously measures and collects data indicative of circadian phase and uses these data to drive the presentation of light of appropriate wavelengths during optimal times in the circadian cycle known to maximize circadian adjustment and sleep quality. Additionally, the data the device collects is self-reported with data from other wireless monitors of sleep quality for periodic examination of cognitive function and decision making to further enhance light presentation.

An integrated solution of circadian rhythm estimation and light-based circadian rhythm adjustment allows effective regulation of circadian rhythms and avoidance of circadian misalignment, leading to improved health, sleep and performance. The present invention includes an optional integrated wearable device coupled with a wireless system that can be utilized as a personal circadian rhythm monitor and regulation device/system capable of rapidly realigning the circadian rhythm of service members to the local environment or, depending on the situation, aligned to provide an artificial environment to ensure both the rhythm of light and user are in sync with the rhythm of activity and sleep, leading to improved sleep and performance. This device and system continuously measures and collects physiological signals, synthesizes them into continuous circadian rhythm estimation, monitors the ambient light to detect circadian misalignments, and controls artificial light presentation. Secure storage of the data set is on the device/system to allow the user and, with proper approval(s), health professionals to perform further evaluation. The data set can include, for example, but not limited to, collected physiological signals, estimated circadian rhythm data, and circadian light monitor control information, as well as user input on self-assessed sleep quality and alertness. The host system can include mobile devices including but not limited to Smart phones, tablets, user/operator control stations or integrations into platform avionics suites and work environments. Integration, portability and interoperability across these platforms and their advanced performance management/training environments are important considerations. The present invention can also be used for SAD and other light therapy applications.

The light sources include light emitting diodes (LEDs) and organic light emitting diodes (OLEDs) and quantum dots (QDs) including ones that are designed to install in conventional legacy light sockets, tombstones, and fixtures, etc. and/or portable light sources. Embodiments of the present invention can be implemented whereby a master coordinator/controller (MCC) communicates with wirelessly-controlled lighting that fits directly into conventional legacy light fixtures (in some embodiments without any changes in the electrical wiring or overhead lighting or lamp design). These LED and OLED lighting sources can change from (non-color) ‘white’ or one or more white color temperatures of light illumination to any color combination of white light plus primary colors such as, but not limited to, red, green, blue (RGB) or red, green, blue, amber (RGBA) or one or more other color temperatures of white depending on the needs indicated by the MCC unit. The MCC or other controllers control features and functions including alarm clock mode, scheduling, synchronization with local time, daylight harvesting and occupancy sensing, etc. These LED and OLED and/or QD light sources are inherently portable, can be fully deployed typically in a time frame of minutes and is easily system integrated to work locations in conjunction with wearable circadian rhythm (CR) devices to provide light feedback for the circadian rhythm regulation and performance systems. In addition they can be rugged, highly reliable, provide controlled dimming and can withstand repeated on/off cycles with no impact on life expectancy. In example embodiments with three color red, green, blue (RGB) or RGB plus amber (RGBA) OLED panels, each individual color can be obtained by turning off the other two colors. To facilitate wake onset and morning circadian phase resetting, a lighting choice with a significant blue color component is selected. To promote sleep onset and permit the nightly evening rise in melatonin a color choice essentially devoid of blue color is selected.

Some embodiments of a circadian rhythm management lighting system include a wearable monitor, LED and/or OLED portable lighting modules or other light sources, and a master coordinator and control unit in direct communication with smart phones, tablets, laptop computers, other computers, etc. Notably, in some embodiments the user can also self-report information using the smart phone/tablet which can also act as an optional way to display circadian rhythm and the circadian rhythm regulation system information and data including for the control and monitoring of the lighting and other environmental information.

The present invention lighting allows virtually any level and ‘size’ of lighting from highly compact lighting that is only a few inches square weighing much less than one pound that can be powered by, for example, batteries to SSL/LED lighting that can be quickly and easily installed in bedrooms, entire houses and apartment buildings to office buildings of practically any size and not limited in size.

Implementations of the present invention allow comparison of circadian rhythm or phase information from commercial off the shelf (COTS) systems whether currently known or developed in the future, as well as devices with well-established markers of circadian phase, including dim light melatonin onset (DLMO) through salivary measures and sleep midpoint analysis.

Implementations of the master coordinator/controller (MCC) wirelessly receive information as input from the circadian rhythm device using any means, including but not limited to WiFi. Bluetooth of all types and flavors, ISM, WeMo, Wink, and Near Field Communications with added channels and/or drivers as desired. The MCC receives signals from smart phones, tablets, laptops, desktops, etc., and the wearable circadian rhythm detection device(s) are in some embodiments able to communicate with, for example, a smart phone, tablet, etc. Sensors, such as cameras and motion detection, can also be used in embodiments of the present invention. Industrial, scientific and medical frequency (ISM) bands and additional sensors as desired can be included in the MCC module. Smart Phone+MCC modules that are portable inexpensive, high powered, optimized can also be used. Software apps can be used to gather, transfer and transmit the pertinent information from the wearable circadian rhythm sensor(s) that is periodically or continuously transmitted to the mobile device and MCC module.

The present invention allows for the ability to integrate, log, archive and catalog data. Data management for collected physiological signals, estimated circadian rhythm, user performance metrics and circadian light modifier control signal information can be used to determine the storage details of how and where the collected physiological signals, estimated circadian rhythm, circadian light control information, the sensor(s) information, the information gathered from the circadian rhythm detector(s), and the control status information along with date, time and location stamps is stored (e.g., in random access memory, non-volatile memory. Flash memory, solid-state drives, USB ‘thumb’ drives, SD cards, hard drives, etc.), hard drives, and other types of storage devices. This information can also be synced up to store on additional mobile devices, PDAs, computers, laptops, etc. to, among other purposes, allow health professionals (with privacy protection) further evaluation.

Example features and functions including, as an example, an alarm clock mode with blue wavelength light content to facilitate waking and to and maximize circadian rhythm phase alignment which could also contain amber wavelength or other wavelengths suitable for use near or at or even during sleep time including in hospital, other care-giving facilities, dormitories, schools, overnight camps, military installations, retirement homes and facilities, convalescent facilities, urgent care facilities, recuperation locations and facilities including temporary, mobile, and permanent ones, etc., combinations of these and other discussed herein, etc.

In some embodiments, timing of light presentation and wavelength can be run through a simulation to determine the anticipated impact on circadian phase based on existing models of human circadian functioning. The MCC can be modified or adjusted accordingly if there is incongruence between the timing of light presentation and the required adjustments in circadian phase.

The white plus color changing lighting or white changing (including, for example, but not limited to, one or more white color temperatures) plus color changing light can be controlled such that, for example, the white and blue LEDs can be selected (enabled) or deselected (disabled) depending on the phase of the circadian rhythm and other measured and available signals and information or selected (enabled) to support SAD or other light therapies.

Existing sensors including daylight harvesting sensors, other photo/light sensors, motion/occupancy sensors, other environment/ambient sensors, etc. can be used with the present invention. The circadian rhythm regulation system can prompt, notify, alert the user if an inappropriate light source such as, for example, a smart phone/tablet or television set is detected that is emitting inappropriate wavelengths for that part/phase of the circadian rhythm cycle. If the user does not respond to the prompts, notifications and/or alerts, the circadian rhythm regulation system will attempt to modify the offending light source to be circadian rhythm cycle phase-compliant. Such prompts can be sent to, among others and not limited to, family, friends, medical staff hospital staff, doctors, care givers, emergency responders, etc. by any means including but not limited to cell phones, land line phones, smart phones, mobile phones, tablets, computers, answering machines, text messages, e-mails, pictures, etc., more than one of these, combinations of these, other methods, ways, etc. discussed herein, etc.

Software apps can be used to gather information including geographical location, time zone, ambient light, settings of in-use digital devices including cell/smart phones, tablets, laptop computers, desktop computer displays and monitors, (if possible) televisions, MP3 players, etc. The system uses this information to adjust the display settings to support circadian rhythm cycle alignment and circadian rhythmicity and to avoid or mitigate circadian desynchrony and circadian disruption as well as treat SAD and provide other types of light therapy.

Embodiments of the present invention can include low-cost portable battery-powered/solar powered optical color ‘notch’ filters so as to be able employ these color filters as and where needed to provide additional optical sensory information and feedback to the MCC unit to aid in circadian rhythm regulation.

Some embodiments of the present invention thus provide a means to improve circadian rhythm, SAD, and other illnesses, diseases, disorders, etc. discussed herein by, for example, but not limited to, providing the appropriate wavelengths of light at appropriate times, based on data from sensors and/or information gathered from various sources and control interfaces, including but not limited to:
Internal and external photosensors including wavelength specific or the ability to gather entire or partial spectrums
Atomic clock(s) signals
Other broadcast time signals
Cellular phone times
Smart phone, tablet, computers, personal digital assistants, etc.
Remote control via dedicated units, smart phones, computers, laptops, tablets, etc.

Embodiments of the present invention include multi-panel configurations including parallel (i.e., same voltage, shared total current through each panel) and series (i.e., same current, stacked voltage). Currently some OLED panels, whether single or multi-color, operate at a total voltage of up to 20+ VDC and are typically connected in parallel and/or series. White-changing OLED panels also provide a certain subset of color changing/tunability. The circadian rhythm lighting and/or SAD and/or light therapy products can use the white-changing/tunable OLED panels to provide blue wavelength enhanced lighting for the ‘wakeup’ and blue wavelength depressed lighting for the ‘sleep-time’ for example, by using layered blue OLEDs and yellow (or amber or orange or similar wavelength color) OLEDs, respectively in any method including layered on top of each other or side-by-side stripes/strips, etc. These respective OLEDs can be color-tuned/turned on, for example, by providing an appropriate current (or in some cases, voltage) to certain electrodes to turn on and excite the proper and desired color or colors depending on the particular point and phase in the circadian rhythm cycle. Implementations of the present invention for both fixed and portable circadian rhythm applications include, but are not limited to, main lighting, under-cabinet and over cabinet lighting for bedrooms, reading rooms, living rooms, dens, family rooms, offices, barracks, hotels, hotel rooms, motel rooms, bed and breakfasts, office buildings, banks, prisons, jails, hospitals of all kinds and types, kitchens, bathrooms, etc., desk, table, task, reading, and portable lamps/lights, accent lamp/lights and special environment lighting and other discussed herein, etc. Some embodiments of the present invention apply multiple floating output current control to driving the respective OLEDs/LEDs/QDs/other forms of SSL, etc., combinations of these, etc.

LEDs, OLEDs, QDs, light sources and panels that are color changing, blue enhanced and blue depressed (for example, but not limited to, orange, amber, yellow, reddish, red, etc.), white changing and special purpose OLEDs can be used for circadian rhythm cycle regulation and assistance and/or SAD and/or other lighting described herein as well as for medical, cleanroom, classroom, nursery, prenatal care, urgent care, long term care, critical care, intensive care, architecture design, etc. and, general lighting, etc.

The present invention applies to OLEDs, LEDs, QDs, other types of SSLs, combinations of these, etc. in general including white and other fixed color, white-changing, color-changing and multi-color, multi-white color temperatures, multi-panel applications including OLEDs of any type including but not limited to stacked, layered, multi-electrode, striped, patterned, etc., OLEDs and edge emitter, edge lit, and waveguided LEDs, QDs, etc.

All of the above can be wirelessly interfaced, controlled and monitored using, for example, smart phones (i.e., iPhones, Androids), tablets (i.e., iPad, iPod touch, droid, etc.), laptops, desktops and other such digital assistants and also other dimming including 0-10 Volt dimming and powerline (PLC) dimming/control. The universal drivers can also support Triac and other forward/reverse phase cut dimming.

In some embodiments of the present invention, the light sources can be easily field replaced and installed without the need to undo anything other than the light source which can be safely removed from the housing and any associated power supplies, drivers, electronics, etc. For example, a RGBW (red green blue white) could be replaced with an RGBA (red green blue amber) or a RBWW (red blue white white), or a RWWW (red white white white) or a WWWW (all white), or an AAAA (all amber), or WAWA (white amber white amber), etc., where the WW, WWW, WWWW can have one or more white color temperatures, etc.

In some embodiments of the present invention, where TVs and displays are used, the present invention either through smart phones or tablets, either directly or through Apps, or a computer or a dedicated interface/controller, remote control, a specially designed remote control, an interface box that allows smart phones, tablets, cell phones, computers, laptops, etc. to communicate through the interface box to control the TV, display, monitor, etc. or combinations of these, etc. can control what is displayed on the screen of the TV or display including a solid color or colors or white or combinations of these (e.g., for wake up white and/or blue, etc.) and solid color or colors such as red, orange, amber, yellow, etc. or combinations of these for sleep time, etc. The wake-up colors could be in different modes that are, for example, but not limited to, being either random and/or synchronized/repeated flashing, color changing, pattern changing, video changing, etc. whereas at sleep time could be sleep-inducing circadian rhythm supporting colors that gradually change and/or fade or dim or turn-off completely after a certain event, amount of time, signal detection, remote commands, wearable and/or other device that detects the physiology, motion, sleep state, temperature, movement, etc. either directly or indirectly, wirelessly or wired, through or with a phone, by hardware, software, firmware of a combination of these, etc. using direct communications or communications via wireless, satellite, cable, internet, phone, web, Apple Healthkit and Homekit, Fitbit, Android products, other wearable products, smart watches, other physiological condition monitors or sensors, and other smart phone and tablet systems, etc., whether gathering measurements directly or based on secondary indications, etc., measuring for example but not limited to heart rate, motion, steps taken, amount of movement, respiration rates, sound level of breathing, body temperature, etc.

Embodiments of the present invention can also control and monitor and switch off the power to, for example, the TV or display at the wall outlet including internal to the wall outlet or external to the wall outlet. Similarly embodiments of the present invention can provide appropriate signals including, but not limited to, video and audio signals to the TV or display to set the color or colors, the pattern of color or colors, the duration of the color or colors, the types of colors, etc. including HGTV, NTSC, PAL, VGA, XVGA, DVI, HDMI, etc. signals.

Embodiments of the present invention include implementations that have amber, yellow, orange, etc., combinations of these, etc. on one side and white, blue-enriched, blue color/wavelength, etc. other wavelengths/colors, etc., combinations of these. In other embodiments of the present invention, for example but not limited to, RGBAW and/or RGBW dies, panels, groups, chips, chip on board, etc., combinations of these can be used to create/support/enhance the appropriate circadian rhythm regulation. The lighting may be OLED, LED, QD, other lighting sources, etc., combinations of these, etc.

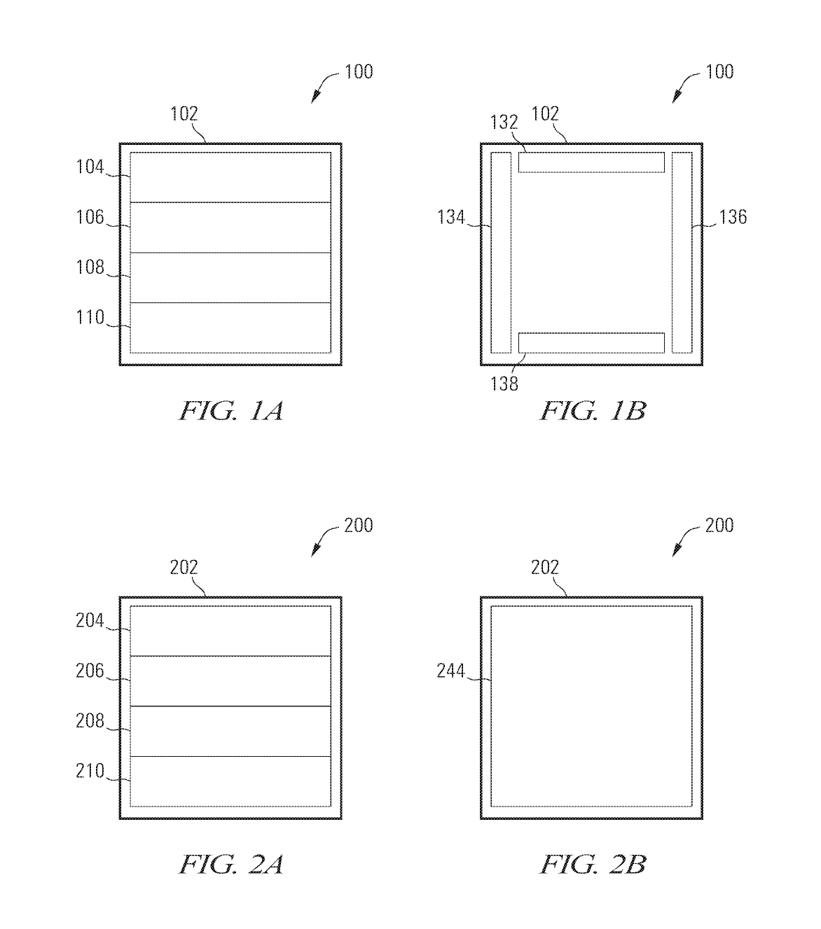

Turning to FIGS. 1A-1B, front and back sides of a solid state lighting panel 100 for use in a circadian rhythm alignment lighting system are depicted in accordance with some embodiments of the invention. In some embodiments, multiple light-emitting elements are mounted on both sides of a frame or substrate 102. The terminology applied to the sides herein, and the orientation of the sides of the panel, is somewhat arbitrary and is not limited to any particular configuration. In some embodiments, the side of the panel 100 shown in FIG. 1A is referred to as the front side if mounted to a wall or sitting on a table, on a floor, etc., or is referred to as the bottom side if mounted to a ceiling, etc. Similarly, the side of the panel 100 shown in FIG. 1B can be referred to as the back side if mounted to a wall or sitting on a table, on a floor, etc., or as the top side if mounted to a ceiling, etc. Multiple light emitting panels, point light sources, arrays of point light sources, etc. can be arranged on the front side of the panel 100 in any suitable configuration, such as an array of OLED or LED light emitting panels 104, 106, 108, 110 that could be yellow, orange, amber etc. or any desired colors or or one or more colors, multiple colors, with light emitting panels 132, 134, 136, 138 in white, blue, etc. on the back side. In some embodiments, the colors emitted on each side of the panel can be used to emit different wavelengths and intensities of light to influence and improve circadian rhythms, for example emitting light during normal waking hours that promotes wakefulness and provides sufficient illumination for task lighting or other normal lighting, and then emitting the same level or dimmer light in wavelengths that promote sleep near the end of normal waking hours. In some embodiments of the present invention, the dimmer light in wavelengths that promote sleep near the end of normal waking hours may continue to become controllably dimmer and dimmer until the light is turned out/off.

In some embodiments, blue and amber OLEDs can be stacked with the blue and amber each having a least one separate electrode, respectively to provide current/power to the respective OLED or both OLEDs, providing the ability to turn on blue light, amber light, or both in a combination to yield a controllable and adjustable white light over a range of color temperatures.

In some embodiments, sensors and/or cameras of any numbers, types, models, functions, etc. are included in lighting panels, enabling monitoring of users or patients undergoing treatment for seasonal affective disorder (SAD) and other types of health issues including but not limited to Alzheimer's, Parkinson disease, mental health problems, Jet Lag or Rapid Time Zone Change Syndrome, Shift Work Sleep Disorder, Delayed Sleep Phase Syndrome (DSPS), Advanced Sleep Phase Syndrome (ASPD), Non 24-Hour Sleep Wake Disorder, etc., combinations of these, etc. Such sensors and/or cameras can determine time periods and/or constancy of gaze of users looking at the lights for treatment periods. Resulting measurements can be recorded, can be provided to users, can be forwarded to treatment providers for review, etc.

Turning to FIGS. 2A-2B, front and back sides of another solid state lighting panel 200 for use in a circadian rhythm alignment lighting system are depicted in accordance with some embodiments of the invention. On one side, multiple light emitting panels 204, 206, 208, 210 that could be yellow, orange, amber etc. or any desired colors are mounted on a substrate 202, with a light emitting panel 244 in one or more white color temperatures, blue, etc. on the back side. In some embodiments, the one or more white color temperatures and/or blue, etc. on the front side with the one or more of yellow, orange, amber, etc. are on the front side. In some embodiments there are also light sources on the sides.

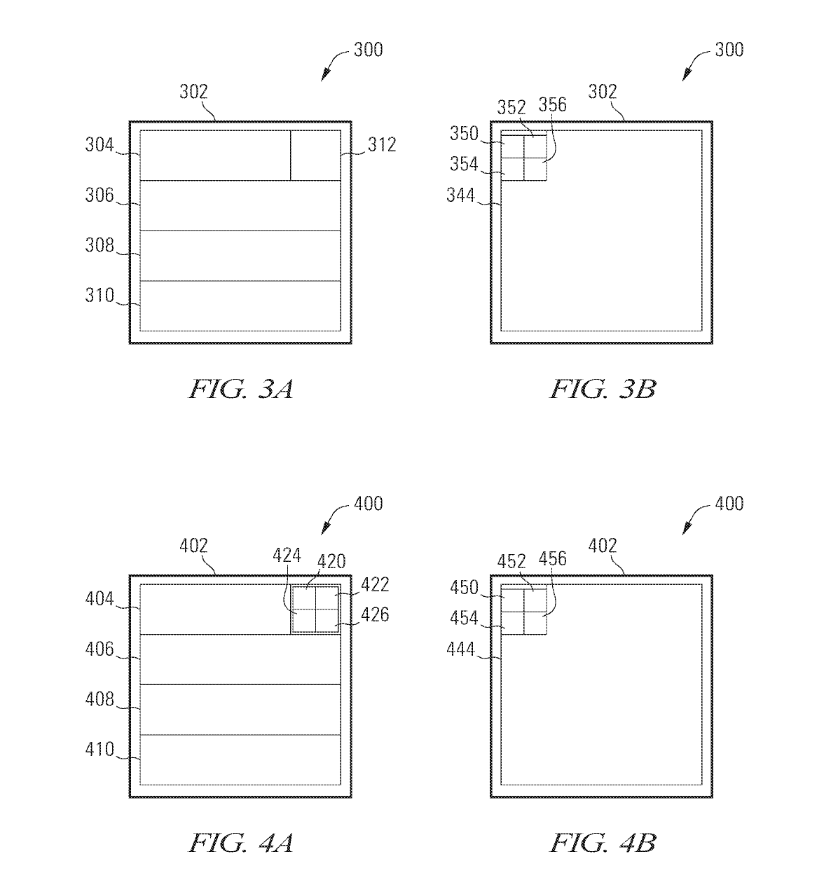

Turning to FIGS. 3A-3B, front and back sides of another solid state lighting panel 300 for use in a circadian rhythm alignment and/or general lighting system are depicted in accordance with some embodiments of the invention. On one side, multiple light emitting panels 304, 306, 308, 310 that could be yellow, orange, amber etc., combined with a blue OLED panel 312, are mounted on a substrate 302, with a light emitting panel 344 in white, blue, etc. on the other side, implemented using one or more OLED panels, combinations of variously colored and/or one or more white color temperature LEDs 350, 352, 354, 356, or in any other suitable manner.

Turning to FIGS. 4A-4B, front and back sides of another solid state lighting panel 400 for use in a circadian rhythm alignment lighting system are depicted in accordance with some embodiments of the invention. On one side, multiple light emitting panels 404, 406, 408, 410 that could be yellow, orange, amber etc., combined with RGB or RGBY or RGBA, etc. LEDs 420, 422, 424, 426, are mounted on a substrate 402, with a light emitting panel 444 in white, blue, etc. on the back side, implemented using one or more OLED panels, combinations of variously colored LEDs 450, 452, 454, 456, or in any other suitable manner.

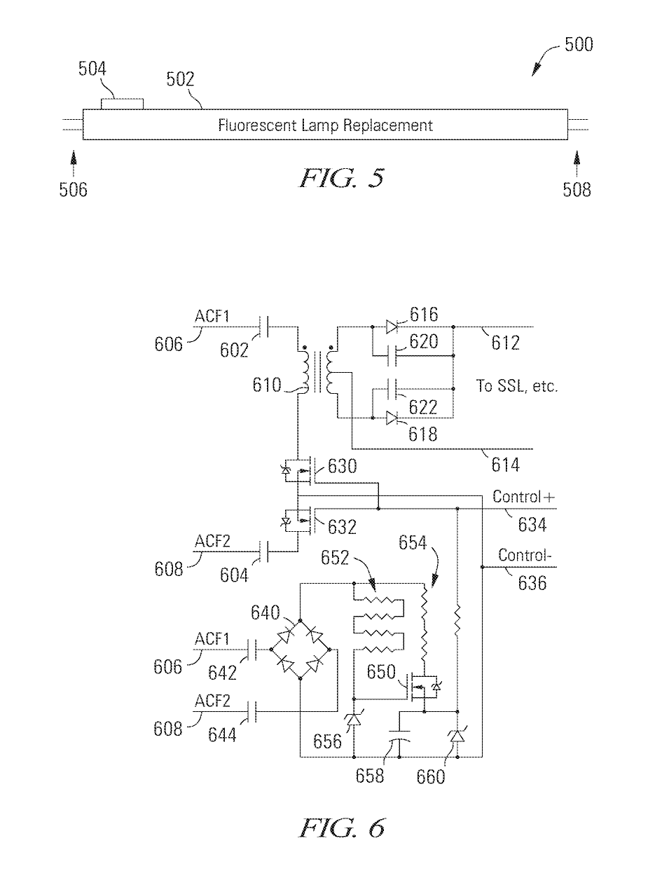

Turning to FIG. 5, in some embodiments of the invention, a solid state fluorescent lamp replacement 500 includes a lamp body 502 with pins 506, 508 enabling it to be connected in a fluorescent lamp fixture. Depending on the type of fixture, any type of electrical connection 506, 508 can be provided, such as, but not limited to, single pins at each end, double pins at each end, or any other configuration. One or more control interfaces/sensors 504 can be provided, supporting analog and/or digital (e.g., 0 to 10 V, 0 to 3 V, 0 to 5 V, 1 to 8 V, DALI, DMX, serial, UART, RS485, RS422, RS232, SPI, I2C, CAN Bus, Modbus, Profibus, DMX512, etc.) or wireless (RF, IR, ISM, Bluetooth, Bluetooth low energy, WiFi, ZigBee, Zwave, IEEE 802, RFID, etc.), or any other type of interface.

Embodiments of the present invention can also have lighting on the outside of, for example, the light bar, panel, etc. including direct lit, edge lit, back lit, etc. Some example embodiments are shown below which can also include one or multiple LEDs, OLEDs. QDs that can consist of one or more of white, red, green, blue, amber, yellow, orange, etc. In addition, such lighting can be used to convey information about the status of a situation including flashing lights which may convey emergency situations, etc.

The lighting may be controlled, dimmed, selected, monitored by wireless (including but not limited to Bluetooth, WiFi, ISM, IEEE 801, 2.4 GHz, etc.) or wired (DMX, DALI, RS 232, RS 485, serial, SPI, U2C, USB, etc.) means.

In other embodiments motors may be used to tilt, swivel, move, move up or down, move left or right, move at an angle, angle, etc. Such embodiments can and may use wireless control of the motors along with the lights, lighting, LEDs, OLEDs, etc.

The present invention can also be used in conjunction with sensors and detectors that determine the emotions and emotional state of a person or persons and respond accordingly including but not limited to, pre-programmed, custom programmed, user-programmed, individual-programmed, universal-response so as to adjust the colors which could include the one or more white color temperatures, CRI, brightness, dimming, the part of or the complete full spectrum, etc. of the lighting to, for example, but not limited to, support the emotional state, help alleviate a negative emotional state, help enhance a positive emotional state, provide a calming environment and ambient, provide a stimulating environment and ambient, etc.

The present invention may be used with people including people with disabilities including hearing impairment, visual impairments, etc., elderly, and others needing assistance by providing a pattern of lighting to, for example, but not limited to, signal someone at the door, the phone ringing, smoke, fire, carbon monoxide, gas, propane, natural gas, other gases, etc., water leakage, temperature changes, humidity changes, time of day/night, etc., combinations of these, by, for example, but not limited to, flashing, color changes, etc., combinations of these which may or may not have audio or sound of any type or form.

The present invention provides a direct replacement for fluorescent tubes used in ballasts and permits dimming even/including if the ballast is not designed to support dimming. Embodiments of the present invention can be powered by either or both a ballast or AC line voltage.

Both wireless and wired control, dimming and monitoring can be accomplished with the present invention. For example wired dimming using 0 to 10 V, 0 to 3 V, 0 to 5 V, or any other voltage range, DALI, can be used or ISM, WiFi, Bluetooth, etc. including in master-slave or other types of groupings where the wired signal is shared by more than one light source which are connected together via wires to provide dimming control and signals.

For example, but not limited to, using 0 to 10V, 0 to 3 V, 0 to 5 V, 1 to 2 V, 1 to 8 V, etc., other analog, DMX, DALI, RS232, RS422, RS485 and other serial and/or parallel interfaces to communicate with the present invention. Some embodiments may Use a connector or connectors to do so. Some embodiments will use an isolated interface.

Embodiments of the present invention can Use, for example, but not limited to, a buck or boost or flyback or forward converter circuit of any type or form including for example, current fed, voltage fed, current controlled, voltage controlled, push-pull, half bridge, full bridge, cuk, SEPIC, etc., that can be powered by AC lines (including universal voltage 80 to 305 VAC, 100 VAC, 120 VAC, 200 VAC, 220 VAC, 240 VAC, 277 VAC, 347 VAC, 480 VAC, etc. at, for example but not limited to, nominally 50/60 Hz) via, in some embodiments, for example, but not limited to an EMI line filter that contains, for example, but not limited to inductors and which also can be powered by an electronic ballast that contains capacitors which limit/block/attenuate/etc. the 50/60 Hz line voltage and bypass (or put in parallel with, etc.) the EMI filter or have the EMI filter on the DC side past the rectification stage.

The present invention can be dimmable and powered on the AC lines or by the ballast.

The present invention can work with dimmable ballasts of any type including but not limited to 0 to 10 V, DALI, TRIAC, powerline control (PLC), etc., instant-start ballasts, rapid start ballasts, programmed start ballasts, programmable start ballasts, pre-start ballasts, magnetic ballasts, and essentially any type of ballast as well as the AC power lines.

The present invention can use a switch, including a momentary switch, for shock hazard protection. For example a momentary switch can be depressed to complete a circuit that allows the ballast to power the present invention once the momentary switch is released. The present invention can also use remote enable to provide protection including protection from shock hazard by essentially keeping the ballast turned off and in a high impedance state until remote commanded (i.e., by remote control, smart phone, tablet, computer, other device, user input, controls/buttons/etc. on the implementations, etc.) to disable the protection/shock hazard. In some preferred embodiments, the user will need to request to disable the protection/shock hazard and then verify/confirm that request to actually disable.

The present invention can use wireless control to control the dimming level of the lighting, etc.

The present invention allows for full spectrum, including full visible spectrum lighting and control, dimming and/or monitoring including red, green, blue (RGB); red, green, blue, amber, (RGBA); red, green, blue, white (RGBW), red, green, blue, amber, white including one or more white color temperatures (RGBAW), additional or fewer colors/wavelengths, etc., combinations of these, etc.

The present invention can use small cards, memories, etc. that can consist of any type of semiconductor memory, magnetic memory, ferromagnetic memory, optical memory, etc., including but not limited to FLASH memory, non-volatile memory, EEPROM, EPROM, PROM, AND memory, OR memory, etc. Such memory can be used to provide programmable information including, for example, but not limited to, ID/name to be used for the present invention, address, individual address, group address, location, properties, behavior, pre-programmed features, data logging, storage of audio and or video information, other information including but not limited to physical, physiological, health, sleep, etc., communications, encryption, type, security, etc.

The present invention, in addition to providing analog and/or digital interfaces for control (including dimming and monitoring, logging, analytics, etc.) can also provide isolated (or non-isolated) power derived from, for example, but not limited to, the ballast itself. An example would be to take current/power from the ballast by rectifying the AC output from the ballast and filtering as desired or needed. As example embodiments which are not intended to be limiting in any way or form include using forward converters or flyback converters for isolated, using buck, boost, buck-boost, boost-buck, etc., linear regulators including current regulators, etc. In some embodiments of the present invention, a keep-alive circuit is used when the present invention is dimmed to very low levels or off.

The present invention can work with all types of sensors and controls including ones that sense movement, proximity, light, solar light, solar energy, daylight, light spectrum(s), temperature, time of day, mechanical, electronic, electrical, sound, vibration, words, voice, voice commands, voice recognition, cell phones, smart phones, tablets, computers, servers, WiFi, Bluetooth, all types and variants of Bluetooth including but not limited to Bluetooth Low Energy, IEEE 802, ISM, serial and/or parallel communications, RFID, entry cards, access cards, signal strength, etc. The present invention can also be used in simple and/or autonomous control and associated modes. Some Implementations may require no external controller or a very simple, easy to use, intuitive one, etc.

Some embodiments of the present invention can also be used to detect the presence (or absence) of a persons or persons including whether a person or persons are spending too much time or too little time in a particular location and, in some embodiments, automatically alert and provide alerts via, for example, but not limited to, e-mail, phone calls, web messages, text messages, lighting changes including flashing lights, blinking, color changing, etc.

The present invention can use parallel or series (or combinations of both) ballasts and can work with or without heater cathodes and can work with magnetic and/or electronic ballasts, etc.

Turning to FIG. 6, an example power supply circuit in a solid state fluorescent lamp replacement for use in a circadian rhythm alignment lighting system is depicted in accordance with some embodiments of the invention. The present invention allows the outputs of one or more (typically two or more) of a ballast to be shared and combined. Capacitors 602, 604 connect the outputs 606, 608 of a ballast, an AC connection or other power source to the primary of a transformer 610 which provides isolated output power across outputs 612, 614 through diodes 616, 618 and capacitors 620, 622, with a center tap of the secondary of the transformer 610 providing a local ground 614. Other embodiments of the present invention may not use a center tapped transformer but may use other types of transformers and, in general, virtually any type of transformer. Embodiments of the present invention can use any type, form, variation, hybrid, etc. of switching or linear voltage or current regulation circuits, topologies, etc.

In series with the primary of the transformer 610 are back to back transistors (switches) 630, 632 which can be used to provide hazard protection by opening up and not allowing a significant current path to exist, controlled by control signal 634, 636. An optional shock hazard protection circuit can also be included, with output connected to control signal 634, 636, and powered by a full or half wave high frequency diode bridge 640 or synchronous rectification stage from the outputs 606, 608 of a ballast, or in some embodiments, an AC line connection or other power source through capacitors 642, 644. The protection circuit can be any type of circuit including but not limited to a half bridge, full bridge, center tapped, etc. An example protection circuit can be powered, for example, using any suitable source or circuit such as a regulator including a transistor 650 and associated resistors 652, 654, Zener diodes 656, 660 and capacitors (e.g., 658) used to open transistors 630, 632 to prevent a significant current path from existing during fault conditions.

There can be multiple such circuits as the one shown above, for example but not limited to, N such circuits for a N-lamp ballast (where typically N=1, 2, 3, 4, 5, 6, etc.) or 2N or 3N or more or less than N.

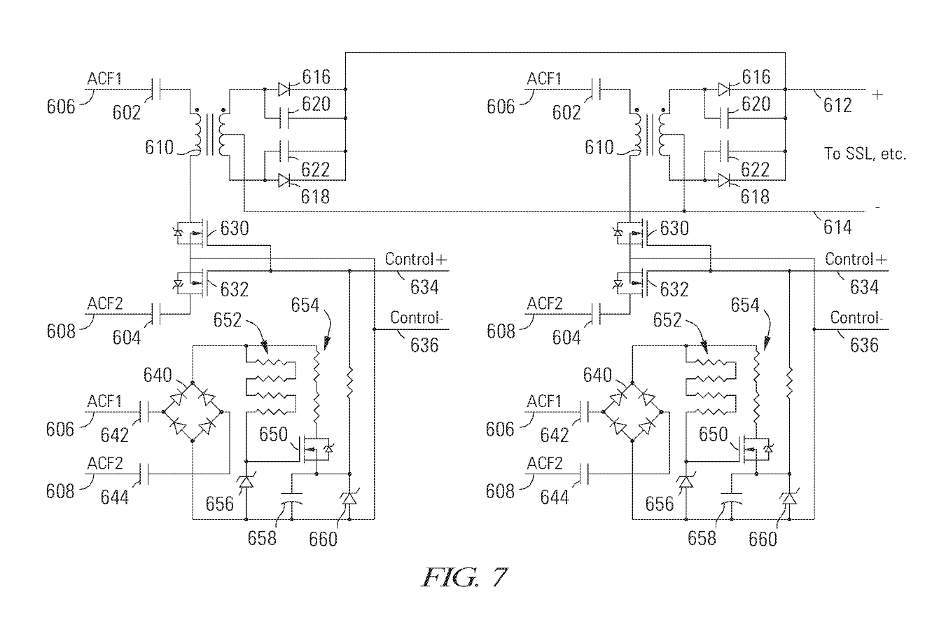

Although only one circuit is shown in FIG. 6, N such circuits can be used in tandem with the outputs tied/connected together, as shown in FIG. 7 in which 2 such circuits are included. In some embodiments of the present invention, more than one output may be desired, needed, etc. in which case, the outputs could be separated or additional power stages and/or power supplies could be incorporated into embodiments and implementations of the present invention.

In some embodiments of the present invention, the combined outputs or separated outputs could be used to drive and supply power to lighting other than fluorescent tubes or fluorescent tube replacements including LED and/or OLED fluorescent tube replacements such as area lighting, color lighting, multi-color lighting, more than one white color temperature, including but not limited to RGBW, RGBAW, RGBWA, RGBWWA, RGBWWWA, full spectrum light, plant growth lighting, smart lighting, growth and health lighting, multi-purpose lighting, special purpose, other light sources, lighting combined with motors, sensors, etc. other type of power requirements, etc. Such light could be of virtually any form factor, shape and size and not confined to merely replacing existing fluorescent tube lighting types and styles. Such lighting could also move, contain motors, be tiltable, tilt, angle, respond to stimuli and information, etc. In addition, in some embodiments of the present invention, the lighting source may be removable and/or interchangeable, replaceable, retrofits, replacements, etc.

Referring to FIG. 6, capacitors 642, 644 and bridge 640 provide raw power to the regulator consisting of resistors 652, 654, Zener diode 656, transistor 650, and capacitor 658 which, in turn, is optionally further limited by Zener diode 660 and supplies gate voltage to protection transistors 630, 632 which are in a back-to-back configuration in series with the primary winding of transformer 610. The secondary of transformer 610 is center tapped with diodes 616, 618 providing full wave rectified power that can be filtered with, for example, optional inductors and capacitors (e.g., 620, 622) and power combined with other similar stages as this one as shown in FIG. 7. The secondary can be at a lower or higher voltage than the primary depending, for example, on the ratio of primary to secondary turns. Less than, equal to, more than N such circuits as above can be powered combined for, for example, an N lamp ballast. In some implementations, less than N such circuits are powered combined for an N lamp ballast. The control + and − signals 634, 636 can be used to override and turn off the back-to-back switches 630, 632.

In some embodiments of the present invention there may be other circuits, functions and features between 612, 614 and before the SSL including but not limited to, one or more diodes, transistors, passive components including capacitor, resistors, inductors, transformers, integrated circuits, etc. that perform one or more of current control, voltage control, power control, under voltage protection, over voltage protection, over current protection, under current protection, under power protection, over power protection, over temperature protection, constant current output, constant voltage output, constant power output, etc., combinations of these, etc.

Again, FIG. 7 shows two such circuits with the output of the respective transformers hooked together to form a combined output where N=2. Although the N=2 case is shown above, N=3, 4, 5, 6 . . . etc., can also be used in various embodiments of the present invention.

In addition, control circuits which either limit the current or the voltage can be used. Such limiting and protection circuits can be, for example but not limited to being, set manually, on time only, by remote control, etc. and also monitored both remotely and locally. Overvoltage, overcurrent, overtemperature, overpower, etc., and other protection and monitoring of faults can be included in the present invention.

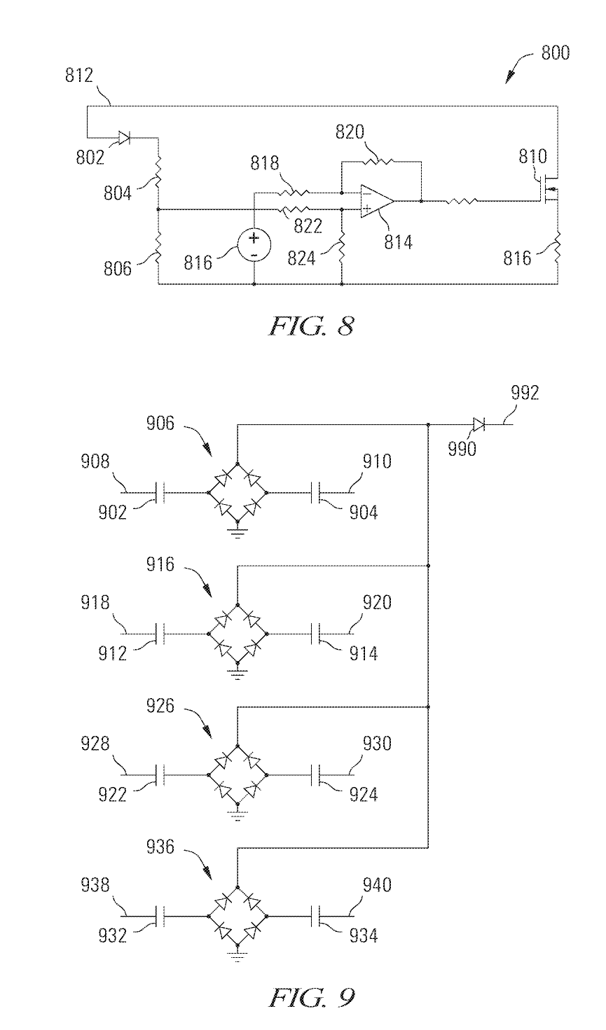

Turning to FIG. 8, a control circuit 800 that can be used to set the output current or voltage of a solid state fluorescent lamp replacement for use in a circadian rhythm alignment lighting system is depicted in accordance with some embodiments of the invention. Diode 802 can be inserted after the full wave center tapped diodes in the isolated versions, and can represent diode 990 in the full wave diode bridge in FIGS. 9-11. The circuit operates by sensing the voltage across voltage divider 804, 806 and turning on transistor 810 to shunt the current from the ballast at node 812 through resistor 816 when the voltage is too high. The inverting input of an op-amp or comparator 814 can be set by any reference voltage 816 and resistors 818, 820. The non-inverting input can be set based on the voltage across voltage divider 804, 806, optionally further divided by voltage divider 822, 824. An example of dimming can be accomplished by locally or remotely (i.e., wired or wireless) changing the reference voltage, Vref, to dim up or dim down the voltage (or current).

Turning to FIG. 9, an example rectifier that can be used in a solid state fluorescent lamp replacement in any form, factor, shape, etc., in a circadian rhythm alignment lighting system is depicted in accordance with some embodiments of the invention. Capacitors 902, 904 along with diode bridge 906 generates a rectified DC output. The input to capacitors 902, 904 is one of the N=4 lamp outputs 908, 910 of the ballast. Likewise the inputs to the other sets of capacitors (i.e., 912 and 914, 922 and 924, 932 and 934) are each fed and supplied by one of the other lamp outputs 918 and 920, 928 and 930, 938 and 940 of the N=4 lamp ballast in this specific example. Diode bridges 916, 926, 936 each form a rectified DC output from each of the lamp outputs of the N=4 lamp ballast in this specific example that is connected together with the output from bridge 906. Thus, for this example but not limited to, the outputs of all 4 of the rectifier stages are tied together to form a common, combined output. In some embodiments of the present invention, some or all the capacitors 902, 904, 912, 914, 922, 924, 932, 934 may be omitted.

In some embodiments, an additional diode 990 is added to allow the excess current (voltage) from the combined output to be, for example but not limited to, shunted on the anode side of the diode 990 by putting a switch across the anode of diode 990 such that the switch is turned on for appropriate amounts of time to shunt the ‘extra’ unwanted current. These amounts of time can be periodic, constant, variable, etc. and can be pulse width modulated (PWM), periodic pulses, frequency modulated, etc., pulses as needed, etc., combinations of these or any other method, technique, approach, for turning on and off a switch including an electronic switch. Capacitor and other filter components and elements may be, for example but not limited to, put on the cathode side 992 of diode 990 to ground.

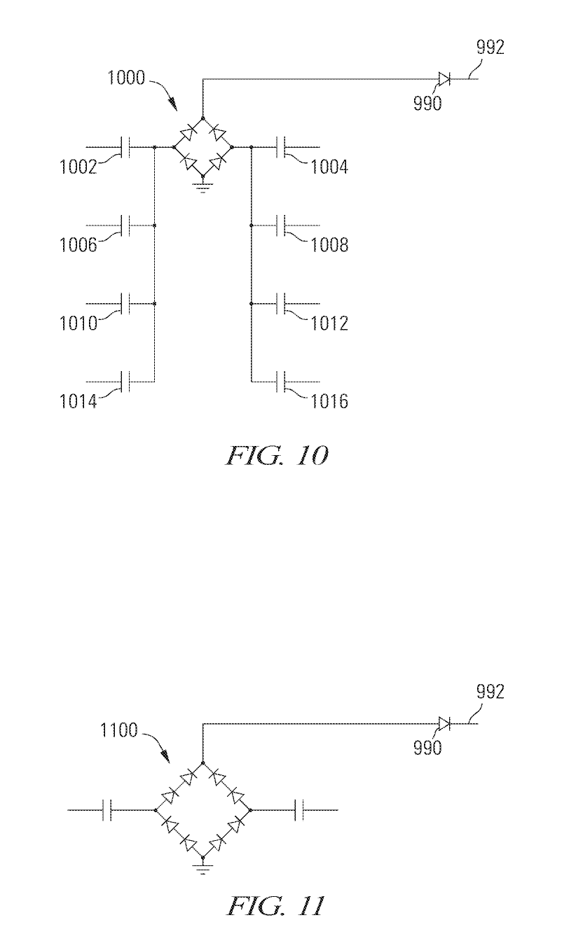

Turning to FIG. 10, a non-isolated power combining/power sharing rectifier that can be used in a solid state fluorescent lamp replacement in a circadian rhythm alignment lighting system is depicted in accordance with some embodiments of the invention. For simplicity, it is assumed that N=4 for the N lamp ballast case although this is merely chosen to illustrate the present invention is not intended to be limiting in any way or form. The choice of more, equal to, less than the number of lamp output s from the ballast can in general be made. A diode bridge 1000 draws power through capacitors 1002, 1004, 1006, 1008, 1010, 1012, 1014, 1016 with each pair (i.e., 1002 and 1004; 1006 and 1008; 1010 and 1012; 1014 and 1016) connected to the respective output of, for example in this particular case, an N=4 lamp ballast and sharing the common full wave bridge 1000 which could consist of, for example, but not limited to, 4 diodes, 6 diodes, 8 diodes, etc. depending, on for example but not limited to, the exact embodiment of the present invention that combines and rectifies the output of the, in this case, N=4 outputs of a N=4 lamp ballast to provide a rectified output from the cathodes of the upper diodes in bridge 1000 to the anode of diode 990 with respect to ground. Diode 990 provides the same function and role as in FIG. 9. That is, diode 990, can, for example, while maintaining the voltage on the cathode side of diode 990, allow the excess current (voltage) from the combined output to be, for example but not limited to, shunted on the anode side of the diode by putting a switch across the anode of diode 990 such that the switch is turned on for appropriate amounts of time to shunt the ‘extra’ unwanted current. These amounts of time and the methodology, topology, approach, etc. can be periodic, constant, variable, etc. and can be pulse width modulated (PWM), periodic pulses, constant on time, constant off time, frequency modulated, etc, pulses as needed, etc., combinations of these or any other method, technique, approach, for turning on and off a switch including an electronic switch. Capacitor and other filter components and elements may be, for example but not limited to, put on the cathode side 992 of diode 990 to ground.

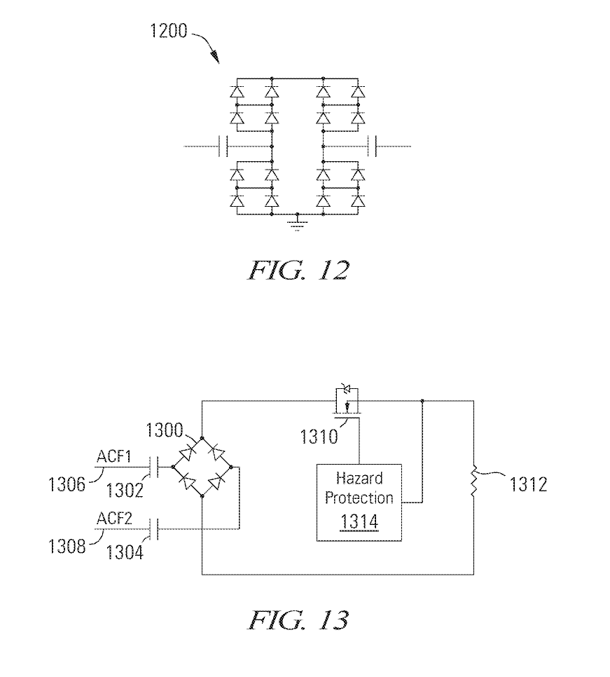

Again, the diode bridge used to rectify power for circadian rhythm alignment lighting can have any suitable topology, configuration, type of components, etc. including but not limited to synchronous rectification, resonant rectification, etc. For example, each leg of a full diode bridge 1100 could consist of, for example, but not limited to, 2 diodes as shown in FIG. 11, or could include 4 diodes, 6 diodes, 8 diodes, etc. Such diode stacked in series could also be used to provide a higher reverse standoff voltage for the full or half bridge implementations, etc. As shown in FIG. 12, each leg of the diode bridge 1200 could also include multiple diodes connected in parallel, in serial, in combinations of parallel and serial connections, etc. to for example, but not limited to, higher current handling capability, higher reverse voltage holdoff, etc. In some embodiments of the present invention, other components including but not limited to resistors, capacitors, etc. may be inserted, for example, in parallel with each of the diodes shown in FIGS. 11 and 12.

Embodiments and implementations of the present inventions allow for dimming, remote control (i.e., wired or wireless) of, for example, but not limited to, the fluorescent lamp replacement using LEDs and/or OLEDs, etc. using analog and/or digital methods, technologies, techniques, interfaces, protocols, specifications, etc.

In addition embodiments of the present invention may be used for other uses, purposes, applications, etc. other than lighting, including but not limited to power supplies, power sources, current sources, general and special application power supplies, etc. power boosters, battery chargers, cell phone chargers, smart phone chargers, computer and tablet power supplies, auxiliary power supplies, cameras including web based, surveillance cameras, fans, heaters, sensors, detectors, etc. of any type, form, use, etc., USB ports, other types of electronics, devices, etc. The sensors and detectors include but are not limited to light, solar, humidity, temperature(s), moisture, spectral and/or precipitation, daylight harvesting, sound, noise, motion, proximity, pressure, speed, occupancy, voice, speech, sound, audio, video, acceleration, biological, physiological, life signs, carbon monoxide, natural gas, other gases, smoke, fire, water, etc.

Embodiments of the present invention can be used with all types of ballasts including but not limited to magnetic, electronic including, but not limited to, instant-start, rapid start, programmable start, programmed start, pre-start, high frequency, low frequency, ballast with cathode heaters, ballasts without cathode heaters, ballasts with starters, ballasts with ignition, etc. In the case of, for example, but not limited to, ballast with heaters, heater emulation circuits can be incorporated into embodiments and implementations of the present inventions. Heater and/or ballast simulation or emulation circuits may comprise any suitable passive and/or active components, such as, but not limited to, resistors and capacitors, etc.

Turning to FIG. 13, a protection mode circuit that can be used in a solid state fluorescent lamp replacement in a circadian rhythm alignment lighting system is depicted in accordance with some embodiments of the invention. A diode bridge 1300 of any configuration is connected through capacitors 1302, 1304 and/or other components to the output 1306, 1308 of a fluorescent fixture or other power source. As with other embodiments, when embodied in a solid state fluorescent lamp replacement, the solid state light power supply can be adapted to operate correctly whether the ballast is in place or has been removed from the fixture and is then powered by, for example, the AC lines or another power supply, etc., regardless of the type of ballast. Of course, the circadian rhythm alignment lighting can use any type of light source and is not limited to use with fluorescent lamp replacements.

A transistor or switch 1310 can be connected across the output of the diode bridge 1300 to shunt the current from the ballast through resistor 1312 when the voltage and/or current is too high. Any hazard detection circuit 1314 or device can be used to control the transistor or switch 1310.

Again, any type of lighting can be used with the circadian rhythm alignment lighting to provide colors, color temperatures, different color temperatures, wavelengths, illumination levels, etc. to improve circadian rhythms, including, but not limited to, computer screens, monitors, panels, etc., tablet screens, smart phone screens, etc., televisions (TVs), LCD and CRT displays of any type or form, DVD and other entertainment lighting and displays containing LEDs, OLEDs, CCFLs, FLs, CRTs, etc., displays, monitors, TVs, OLED, LED, CCFL, FL, incandescent lighting, etc.

Low voltage (12 V) AC and DC lighting systems and components including MR16, GU10, etc. can also be used for the present invention including RGBW, RGBWW, RGBWWA and the use of RGBW—i.e., R and G to produce yellow for night time, sleep time, sleep, etc. mode and BW (blue white where white can consist of one or more white color temperatures) to produce light suitable for wake up mode—which can be in any form and could include but is not limited to a wireless or wired or powerline control (PLC) receiver, transceiver, transmitter, etc. Although a low voltage MR16 was discussed, the present invention also equally applies to all types and forms of general lighting including, but not limited to, GU10, A-lamps, E26 socket lighting, E27 socket lighting, PAR30, PAR38, R30, R40, T12, T10, T9, T8, T5, T4, etc. and other types and forms of LED lighting, etc.

The RGBW, RGBWW, RGBA, RGBWWA, etc. can consist of discrete LEDs or packaged LEDs of any size and form and also could consist of additional colors and quantities such as RGBWA, RGBWB, RGBWA, RGBWWA, etc.

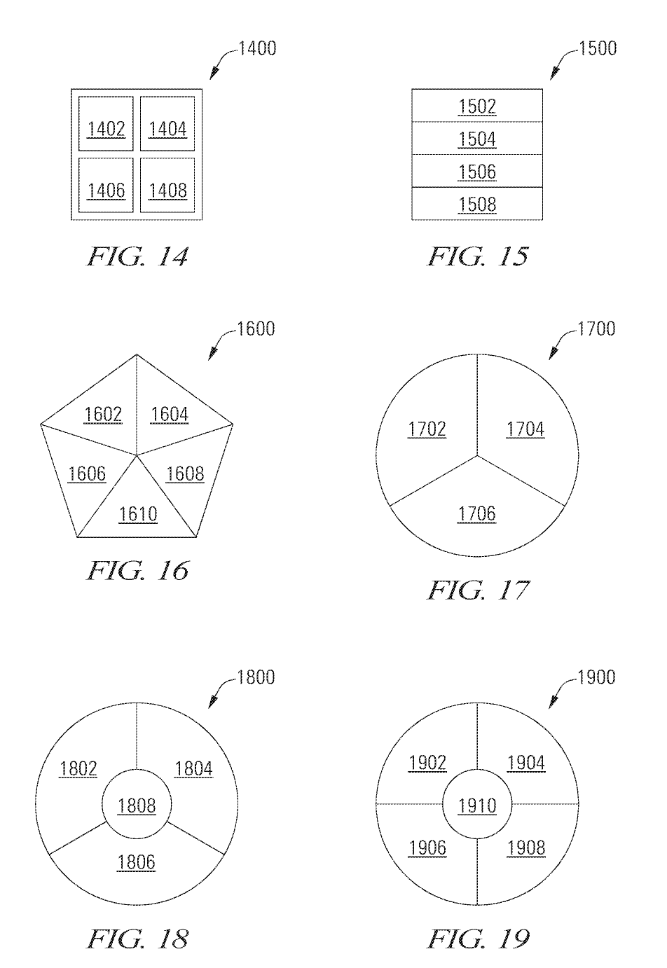

The present invention also includes dies of any type and form and arrangement that consists of four or more LEDs in which one of the LEDs is white—again, for example, RGBW, RGBWA (or RGBAW, RGBWWA, etc.). The package, substrate, die, etc. that the four or more LEDs with one LED being white (e.g., RGBW) include plastic, ceramic, composite, polymers, metal, etc., combinations of these, etc. The ceramic(s) can be of any type including but not limited to oxides, nitrides, etc. such as aluminum oxide, sapphire, quartz, aluminum nitride, beryllium oxide, boron nitride, etc. Some simple examples of LED packages of the present invention include, but are not limited to those shown in FIGS. 14-19. Any shape can be used including essentially round, square, rectangular, elliptical, parabolic, semi-circle, semi-sphere, sphere and other standard and non-standard essentially 2 and 3 dimensional shapes and forms, etc. Two or more wires/pads/pins/etc. may be used per LED color or some wires/pads/pins/etc. may be reduced to reduce count, etc. As shown in FIG. 14, an example LED light package 1400 can have a square or rectangular shape and can include any number of LEDs and colors, such as, but not limited to, red 1402, green 1404, blue 1406 and white 1408. As shown in FIG. 15, another example LED light package 1500 can include light emitting panels or strips such as, but not limited to, red 1502, green 1504, blue 1506 and white 1508. As shown in FIG. 16, another example LED light package 1600 can include light emitting panels or strips such as, but not limited to, red 1602, green 1604, blue 1606, yellow or amber 1608 and white 1610. As shown in FIG. 17, another circular example LED light package 1700 can include light emitting sections such as, but not limited to, red 1702, green 1704, blue 1706. As shown in FIG. 18, another circular example LED light package 1800 can include light emitting sections such as, but not limited to, red 1802, green 1804, blue 1806, white 1808. As shown in FIG. 19, another circular example LED light package 1900 can include light emitting sections such as, but not limited to, red 1902, green 1904, blue 1906, yellow or amber 1908, and white 1910. All of these may also contain more than one white color temperature LEDs.

For any of the present inventions discussed herein, power supplies of any type, form, topology, architecture, etc. including but not limited to non-isolated and/or isolated power supplies and drivers such as buck, buck-boost, boost-buck, boost, Cuk, SEPIC, forward converters, push-pull, current mode, voltage mode, current fed, voltage fed, one-stage, two-stage, multi-stage, high power factor, linear, switching, etc. may, in general, be used.

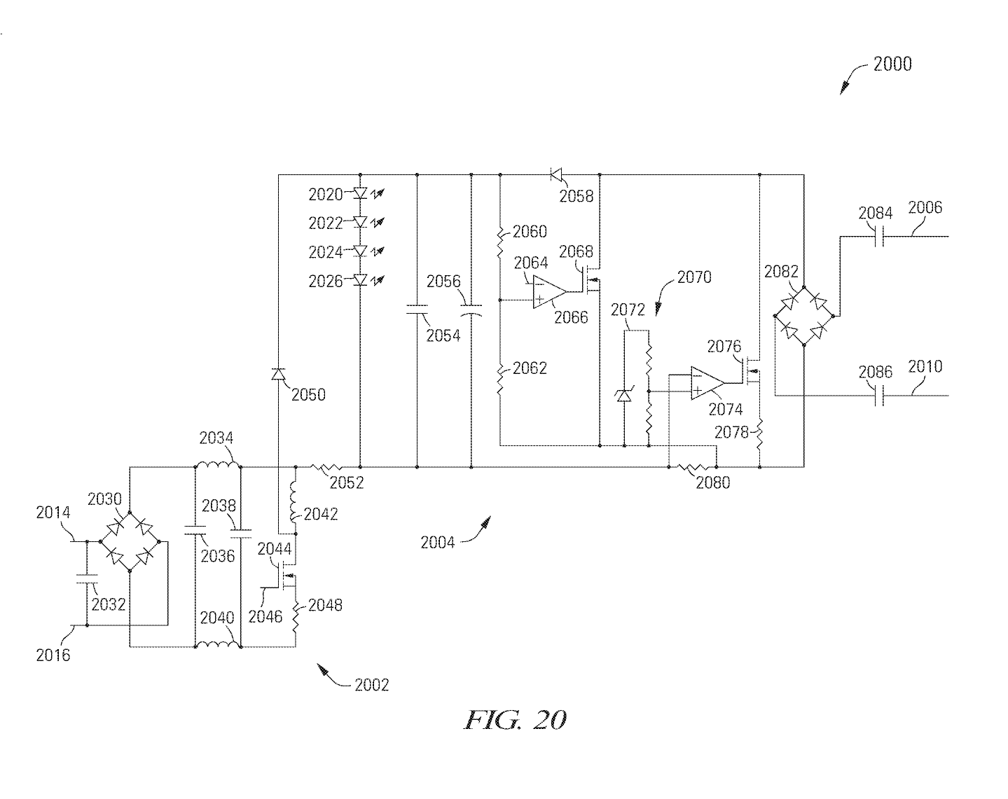

Turning to FIG. 20-22, an example power supply circuit 2000 with various input connections in a solid state fluorescent lamp replacement for use in a circadian rhythm alignment lighting system is depicted in accordance with some embodiments of the invention. The power supply circuit 2000 is adapted for connection in a fluorescent lamp fixture, either with or without ballast in place, to power one or more solid state lights (e.g., 2020, 2022, 2024, 2026). Depending on whether a ballast is present in the fixture, the power supply circuit 2000 can supply power through an AC supply circuit 2002 or a ballast supply circuit 2004. The power supply circuit 2000 can be used with any type of fluorescent light fixture, and with any type of ballast or with no ballast, including with multiple output ballasts, or with multiple ballasts in the fixtures.

The AC supply circuit 2002 is not limited to the example circuit shown in FIG. 20. In some example embodiments, the AC supply circuit 2002 includes a full diode bridge rectifier 2030 connected to AC input 2014, 2016. A capacitor 2032 can be included which can be used on the AC line as part of an EMI filter and when powered by a ballast as a way to reduce the maximum ballast voltage including when the circuit is drawing no or very little current/power from the ballast, as well as other example but not limiting optional EMI filter components such as, but not limited to, inductors 2034, 2040 and capacitors 2036, 2038. The example AC supply circuit 2002 regulates current to the load 2020-2026 by, for example, pulse width modulation of transistor or switch 2044. A storage inductor 2042 or switching inductor stores energy when transistor 2044 is on and current flows from the input 2014, 2016 through inductor 2042 and load 2020-2026. A switching diode 2050 or buck diode provides a current path when transistor 2044 is off, allowing energy stored in inductor 2042 to flow through load 2020-2026. Again, the AC supply circuit 2002 is not limited to use with the buck circuit of FIG. 20 or for use with any particular circuit or topology. Although a buck converter circuit is depicted in FIGS. 20 to 22, In various embodiments, the AC supply circuit 2002 can be implemented using buck circuits, buck-boost, boost-buck, boost, flyback, Cuk, SEPIC, forward converters, push-pull, current mode, voltage mode, current fed, voltage fed, one-stage, two-stage, multi-stage, high power factor, switching, linear, resonant converters, half bridge, full bridge, etc., combinations of these, etc., hybrids of these, etc. Any or all of these circuit topologies for 2002 can have, for example but not limited to over current protection, over voltage protection, under voltage protection, over temperature protection, etc. In addition 2002 can be locally or remotely dimmable including digitally dimmable and triac/wall/forward or reverse phase dimmable, remotely dimmable using analog or digital control via wired, wireless, powerline communications, etc. control and monitoring including performing remote diagnostics and analytics as well as tracking power usage, power control, sensor detection, etc. In general any form of dimming can be used including but not limited to PWM dimming, analog dimming, digital dimming, etc.

The ballast input supply circuit 2004 includes a high frequency capable full diode bridge rectifier 2082 connected to ballast inputs 2006, 2010 through series capacitors 2084, 2086. When a ballast is in place, rectified current flows from the rectifier 2082 through diode 2058 through load 2020-2026.

Capacitors (e.g., 2054, 2056) can be connected in parallel with load 2020-2026 or in any suitable manner, as desired, to smooth the current through the example load 2020-2026 (which could be made of any number of, for example LEDs and/or OLEDs in series or parallel or both covering a relatively large range of forward LED/OLED voltages from ˜3 volts to well over 100 V and higher) into a DC current, and are shared by both the AC supply circuit 2002 and the ballast input supply circuit 2004. The ballast input supply circuit 2004 can also perform current control, over-voltage protection, over-temperature protection, and any other protection and control features desired. For example, comparator 2064 can be included to provide over-voltage protection, comparing a reference voltage at the inverting input 2064 with an output voltage divided in voltage divider 2060, 2062. When the output voltage rises above a threshold, the comparator 2066 turns on transistor 2068, effectively shorting across the ballast inputs 2006, 2010 through rectifier 2082 to limit the output voltage.

Comparator 2074 can be included to control the output current, comparing the voltage across a sense resistor 2080 with a reference voltage provided by a voltage reference source 2070. When the output current rises to a level at which the voltage across the sense resistor 2080 is greater than the reference voltage, the comparator 2074 turns on transistor 2076, effectively shorting across the ballast inputs 2006, 2010 through rectifier 2082 until the current drops below the threshold and transistor 2076 is turned back off or, for example, but not limited to, for a set or prescribed time or a programmed time duration, etc. The voltage reference source 2070 can be implemented in any suitable manner, such as, but not limited to, a Zener diode connected in series with a pullup resistor (not shown), and with a voltage divider sampling the voltage between the Zener diode and the pullup resistor to generate the reference voltage at the non-inverting input of the comparator 2074. In general, any type of voltage reference can be used including but not limited to bandgap references, voltage regulators including but not limited to linear voltage regulators, etc. In addition 2000 can be locally or remotely dimmable including digitally dimmable, remotely dimmable using analog or digital control via wired, wireless, powerline communications, etc. control and monitoring including performing remote diagnostics and analytics as well as tracking power usage, power control, sensor detection, etc. and, under some instances, triac/wall/forward or reverse phase dimmable, In general any form of dimming can be used including but not limited to PWM dimming, analog dimming, digital dimming, etc.

The example power supply circuit 2000 has been shown in a simplified manner for clarity. Other components can be included as needed and desired. For example, a pulse generator (not shown) can be used to generate a pulse control signal at the gate of transistor 2046, such as, but not limited to, a 100 kHz signal or any other frequency with varying pulse width. In some embodiments, the gate of transistor 2046 can be driven, for example, from a rectified signal derived from the ballast input through a diode. In some embodiments, the signal driving the gate of transistor 2046 can be switchable between a pulse generator output and a rectified signal derived from the ballast input, or by a control signal generated by any other controller or source. Current control and/or dimming can also be implemented, for example, based on output current measurement using a sense resistor 2052 to control the pulse signal operating transistor 2044. For example, measuring the voltage across sense resistor 2052 from a local ground between resistors 2052, 2080 to the node between sense resistor 2052 and inductor 2042 yields a negative voltage, which can be used in a summing circuit to compare to a positive reference voltage to set the output current level. An opto-coupler or other device(s), circuit, component, etc., can be used to level-shift and isolate the resulting feedback signal, which is then used to control a pulse generator to modulate transistor 2044 to yield the desired output current level. Any form or type of operation can be used including discontinuous conduction mode (DCM), continuous conduction mode (CCM), critical conduction mode (CRM), resonant conduction mode (RCM), etc., combinations of these, etc.

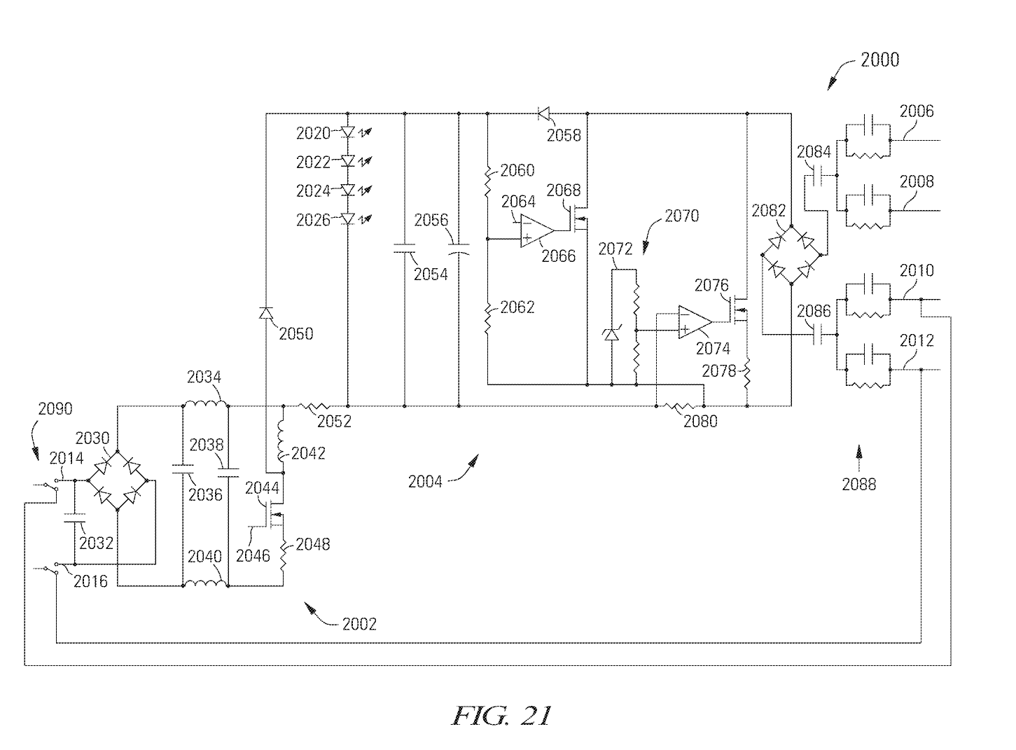

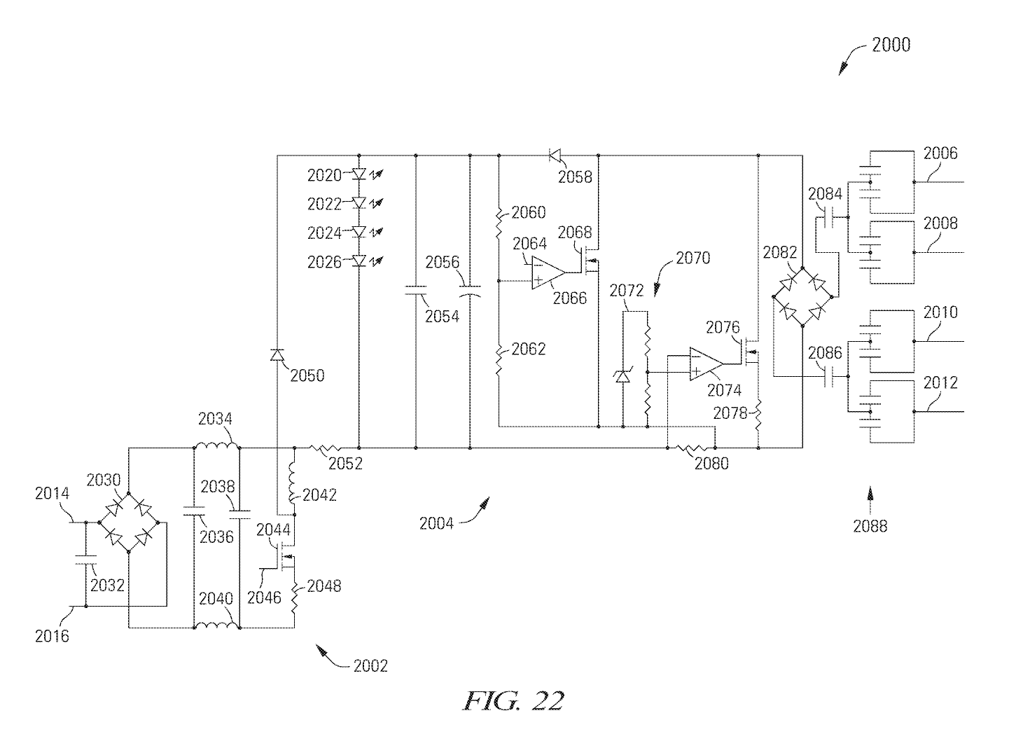

The example power supply circuit 2000 can be connected in a fluorescent lamp replacement in a variety of configurations. For example, but not limited to, the ballast inputs 2006, 2010 can be connected to a pin at either end of a fluorescent lamp fixture, with AC inputs 2014, 2016 connected to the same pins at one end. As shown in FIG. 21, in some embodiments the example power supply circuit 2000 includes heater simulation circuits such as, but not limited to, the parallel combination of capacitor and resistor connected to both bi-pin inputs 2006, 2008, 2010, 2012 at the ends of a fluorescent lamp fixture. In some embodiments, optional switches 2090 can be included to switch between ballast input and AC input, such that the AC supply circuit 2002 is either powered from AC inputs or ballast inputs; however in general these are optional switches if desired. As shown in FIG. 22, in some embodiments, parallel capacitors 2088 at the input to the ballast input supply circuit 2004 provide symmetry so that there is no need to distinguish between heater pins and beam pins at the end of bi-pin fluorescent lamp fixtures.

In some embodiments, wired or wireless control signal(s) can be used to control and/or dim the power supply circuit 2000, for example controlling reference voltages such as that at the inverting input 2064 of comparator 2066. Such a control signal can be received and processed using any suitable circuits and types of signals, including but not limited to Bluetooth of any type or flavor including Bluetooth, Bluetooth low energy, WiFi, IEEE 801, IEEE 802, ZigBee, Zwave, other 2.4 GHz and related/associated standards, protocols, interfaces, RFID, ISM, other frequencies including but not limited to, radio frequencies (RF), microwave frequencies, millimeter-wave frequencies, sub millimeter-wave frequencies, terahertz (THz), mobile cellular network connections, combinations of these, serial or parallel interfaces and/or protocols of any type or form including those discussed herein such as but not limited to 0 to 10V, 0 to 3 V, other voltages, DMX, DALI, RS485, SPI, I2C, etc. In addition, voice commands, voice recognition, voice detection, proximity, time of day, and/or other signals or conditions can be used to control the power supply circuit 2000. In some embodiments, 2020 through 2026 may represent more than one color LEDs and/or OLEDs, more than one white color temperature LEDs or OLEDs, QDs, combinations of these in series, parallel, etc.

In some embodiments of the present invention, 2014 may be connected in parallel with, for example but not limited to, 2006 and 2016 may be connected in parallel with 2010 in FIG. 20. In other embodiments of the present invention, 2014 may be connected in parallel with, for example but not limited to, 2010 and 2016 may be connected in parallel with 2006, etc. in FIG. 20.

Although 2010 and 2012 are shown connected to optional switches 2014 and 2016 in FIGS. 21, 2006 and 2008 could instead in place of 2010 and 2012 be connected to optional switches 2014 and 2016 in FIG. 21,

In some embodiments of the present invention, 2014 may be connected in parallel with, for example but not limited to, 2006 and 2016 may be connected in parallel with 2008 in FIG. 22. In other embodiments of the present invention, 2014 may be connected in parallel with, for example but not limited to, 2010 and 2016 may be connected in parallel with 2012, etc. in FIG. 20.

Additional diodes and other passive and active components including but not limited to transistors may be added as desired or needed.

In some embodiments of the present invention, one or more inductors (as well as and/or in addition to capacitors and other passive and active elements) can be used. With capacitance on the output, capacitance can also be placed on the input to cut down on spurious signals including noise and spikes. In some embodiments of the present invention, an inductor can also be put in series with switches and diodes, and a clamp diode to contain the flyback voltage. In some embodiments of the present invention, inductors can be put on either or both the input and/or output to also provide filtering to reduce the ripple to the load (i.e., LED and/or OLED array). The switching frequency of, for example, Q1 could typically be in the range of 20 kHz or higher (i.e., typically above the human audio range) or, in the case of overcurrent or overvoltage conditions, possibly lower and even much lower than 20 kHz or higher. For dimming, the frequency of the PWM dimming may be much lower and typically in the range of ˜100 Hz and higher. For PWM switching and PWM dimming switching, switching can be done on either side of, for example, a transformer for embodiments of the present invention depending on considerations that, for example, determine the appropriate placement.

Embodiments of the present invention allow for no, passive and/or active control. Some embodiments of the present invention provide in the matching circuit, for example, a chopper that typically can be switching in a frequency range of less than 20 kHz to greater than 100 kHz, either free running, self-oscillating or controlled, so that the transformer can be small even with a 60 Hz ballast. In addition, by providing a regulator circuit, in some embodiments it can make the LED independent of the ballast, and therefore universal.

In some embodiments of the present invention, the PWM converter used for the series regulation from the AC lines can also be used for the shunt regulation from the ballast output, with the control inverted from a normal voltage-in/voltage-out converter or voltage-in/current-out operation.

With a ballast, some implementations of the present invention utilize current output control with a shunt regulator with switching mode regulation chosen. In this case, the regulator switches to effective/local ground (low voltage drop equals low power dissipation) or open (no current equals low power dissipation). In addition to the passive and active components mentioned previously, other protection and detection devices and components can be used with the present invention including but not limited to tranzorbs, transient voltage suppressors (TVSs), varistors, metal oxide varistors (MOVs), surge absorbers, surge arrestors, and other transients detection and protection devices, thermistors or other thermal devices, fuses, resettable fuses, circuit breakers, solid-state circuit breakers and relays, other types of relays including mechanical relays and circuit breakers, etc.

In embodiments of the present invention that include or involve buck, buck-boost, boost, boost-buck, etc. inductors, one or more tagalong inductors such as those disclosed in U.S. patent application Ser. No. 13/674,072, filed Nov. 11, 2012 by Sadwick et al. for a “Dimmable LED Driver with Multiple Power Sources”, which is incorporated herein for all purposes, may be used and incorporated into embodiments of the present invention. Such tagalong inductors can be used, among other things and for example, to provide power and increase and enhance the efficiency of certain embodiments of the present invention. In addition, other methods including charge pumps, floating diode pumps, level shifters, pulse and other transformers, bootstrapping including bootstrap diodes, capacitors and circuits, floating gate drives, carrier drives, etc. can also be used with the present invention.

Programmable soft start including being able to also have a soft short at turn-on which then allows the input voltage to rise to its running and operational level can also be included in various implementations and embodiments of the present invention.

Some embodiments of the present invention utilize high frequency diodes including high frequency diode bridges and current to voltage conversion to transform the ballast output into a suitable form so as to be able to work with existing AC line input PFC-LED circuits and drivers. Some other embodiments of the present invention utilize high-frequency diodes to transform the AC output of the electronic ballast (or the low frequency AC output of a magnetic ballast into a direct current (DC) format that can be used directly or with further current or voltage regulation to power and driver LEDs for a fluorescent lamp replacement. Embodiments of the present invention can be used to convert the low frequency (i.e., typically 50 or 60 Hz) magnetic ballast AC output to an appropriate current or voltage to drive and power LEDs using either or both shunt or series regulation. Some other embodiments of the present invention combine one or more of these. In some embodiments of the present invention, one or more switches can be used to clamp the output compliance current and/or voltage of the ballast. Various implementations of the present invention can involve voltage or current forward converters and/or inverters, square-wave, sine-wave, resonant-wave, etc. that include, but are not limited to, push pull, half-bridge, full-bridge, square wave, sine wave, fly-back, resonant, synchronous, etc.

For the present invention, in general, any type of transistor or vacuum tube or other similarly functioning device can be used including, but not limited to, MOSFETs, JFETs, GANFETs, depletion or enhancement FETs, N and/or P FETs, CMOS, PNP BJTs, triodes, etc. which can be made of any suitable material and configured to function and operate to provide the performance, for example, described above. In addition, other types of devices and components can be used including, but not limited to transformers, transformers of any suitable type and form, coils, level shifters, digital logic, analog circuits, analog and digital, mixed signals, microprocessors, microcontrollers, FPGAs, CLDs, PLDs, comparators, op amps, instrumentation amplifiers, and other analog and digital components, circuits, electronics, systems etc. For all of the example figures shown, the above analog and/or digital components, circuits, electronics, systems etc. are, in general, applicable and usable in and for the present invention. In some embodiments of the present invention that use transformers, the transformers may be implemented with extra windings to provide current sensing, current feedback, bias supplies, auxiliary supplies, logic supplies, isolated supplies, isolated regulated supplies for sensors, cameras, battery chargers, etc.

The example figure and embodiments shown in FIGS. 1 through 22 are merely intended to provide some illustrations of the present inventions and not limiting in any way or form for the present inventions.

Using digital and/or analog designs and/or microcontrollers and/or microprocessors any and all practical combinations of control, sequencing, levels, etc., some examples of which are listed below for the present invention, can be realized.

In addition to these examples, a potentiometer or similar device such as a variable resistor may be used to control the dimming level. Such a potentiometer may be connected across a voltage such that the wiper of the potentiometer can swing from minimum voltage (i.e., full dimming) to maximum voltage (i.e., full light). Often the minimum voltage will be zero volts which may correspond to full off and, for the example embodiments shown here, the maximum will be equal to or approximately equal to the voltage on the negative input of, for example, a comparator or op amp. Current sense methods including resistors, current transformers, current coils and windings, etc. can be used to measure and monitor the current of the present invention and provide both monitoring and protection.

In addition to dimming by adjusting, for example, a potentiometer, the present invention can also support all standards, ways, methods, approaches, techniques, etc. for interfacing, interacting with and supporting, for example, 0 to 10 V or 0 to 3 V or some other range of analog input dimming by, for example, replacing a suitable reference voltage that can be remotely set or set via an analog or digital input such as illustrated in patent application 61/652,033 filed on May 25, 2012, for a “Dimmable LED Driver”, which is incorporated herein by reference for all purposes.

The present invention supports all standards and conventions for 0 to 10 V or 0 to 3 V dimming or other dimming techniques. In addition the present invention can support, for example, overcurrent, overvoltage, short circuit, and over-temperature protection. The present invention can also measure and monitor electrical parameters including, but not limited to, input current, input voltage, power factor, apparent power, real power, inrush current, harmonic distortion, total harmonic distortion, power consumed, watthours (WH) or killowatt hours (kWH), etc. of the load or loads connected to the present invention. In addition, in certain configurations and embodiments, some or all of the output electrical parameters may also be monitored and/or controlled directly for, for example, LED drivers and FL ballasts. Such output parameters can include, but are not limited to, output current, output voltage, output power, duty cycle. PWM, dimming level(s), etc.

In place of the potentiometer, an encoder or decoder can be used. The use of such also permits digital signals to be used and allows digital signals to either or both locally or remotely control the dimming level and state. A potentiometer with an analog to digital converter (ADC) or converters (ADCs) could also be used in many of such implementations of the present invention. The examples and figures herein are merely meant to provide illustrations of the present and should not be construed as limiting in any way or form for the present invention.

In addition to the examples above and any combinations of the above examples, the present invention can have multiple dimming levels set by the dimmer in conjunction with the motion sensor and photosensor/photodetector and/or other control and monitoring inputs including, but not limited to, analog (e.g., 0 to 10 V, 0 to 3 V, etc.), digital (RS232, RS485, USB, DMX, SPI, SPC, UART, other serial interfaces, etc.), a combination of analog and digital, analog-to-digital converters and interfaces, digital-to-analog converters and interfaces, wired, wireless (i.e., RF, WiFi, ZigBee, Zwave, ISM bands, 2.4 GHz, etc.), powerline (PLC) including X-10, Insteon, HomePlug, etc.), etc.

The present invention is highly configurable and words such as current, set, specified, etc. when referring to, for example, the dimming level or levels, may have similar meanings and intent or may refer to different conditions, situations, etc. For example, in a simple case, the current dimming level may refer to the dimming level set by, for example, a control voltage from a digital or analog source including, but not limited to digital signals, digital to analog converters (DACs), potentiometer(s), encoders, etc.

The present invention can have embodiments and implementations that include manual, automatic, monitored, controlled operations and combinations of these operations. The present invention can have switches, knobs, variable resistors, encoders, decoders, push buttons, scrolling displays, cursors, etc. The present invention can use analog and digital circuits, a combination of analog and digital circuits, microcontrollers and/or microprocessors including, for example, DSP versions, FPGAs, CLDs, ASICs, etc. and associated components including, but not limited to, static, dynamic and/or non-volatile memory, a combination and any combinations of analog and digital, microcontrollers, microprocessors, FPGAs, CLDs, etc. Items such as the motion sensor(s), photodetector(s)/photosensor(s), microcontrollers, microprocessors, controls, displays, knobs, etc. may be internally located and integrated/incorporated into the dimmer or externally located. The switches/switching elements can consist of any type of semiconductor and/or vacuum technology including but not limited to triacs, transistors, vacuum tubes, triodes, diodes or any type and configuration, pentodes, tetrodes, thyristors, silicon controlled rectifiers, diodes, etc. The transistors can be of any type(s) and any material(s)—examples of which are listed below and elsewhere in this document.

The dimming level(s) can be set by any method and combinations of methods including, but not limited to, motion, photodetection/light, sound, vibration, selector/push buttons, rotary switches, potentiometers, resistors, capacitive sensors, touch screens, wired, wireless, PLC interfaces, etc. In addition, both control and monitoring of some or all aspects of the dimming, motion sensing, light detection level, sound, etc. can be performed for and with the present invention.

Other embodiments can use other types of comparators and comparator configurations, other op amp configurations and circuits, including but not limited to error amplifiers, summing amplifiers, log amplifiers, integrating amplifiers, averaging amplifiers, differentiators and differentiating amplifiers, etc. and/or other digital and analog circuits, microcontrollers, microprocessors, complex logic devices (CLDs), field programmable gate arrays (FPGAs), etc.

The dimmer for dimmable drivers may use and be configured in continuous conduction mode (CCM), critical conduction mode (CRM), discontinuous conduction mode (DCM), resonant conduction modes, etc., with any type of circuit topology including but not limited to buck, boost, buck-boost, boost-buck, cuk, SEPIC, flyback, forward-converters, etc. The present invention works with both isolated and non-isolated designs including, but not limited to, buck, boost-buck, buck-boost, boost, cuk, SEPIC, flyback and forward-converters. The present invention itself may also be non-isolated or isolated, for example using a tagalong inductor or transformer winding or other isolating techniques, including, but not limited to, transformers including signal, gate, isolation, etc. transformers, optoisolators, optocouplers, etc.

The present invention may include other implementations that contain various other control circuits including, but not limited to, linear, square, square-root, power-law, sine, cosine, other trigonometric functions, logarithmic, exponential, cubic, cube root, hyperbolic, etc. in addition to error, difference, summing, integrating, differentiators, etc. type of op amps. In addition, logic, including digital and Boolean logic such as AND, NOT (inverter), OR. Exclusive OR gates, etc., complex logic devices (CLDs), field programmable gate arrays (FPGAs), microcontrollers, microprocessors, application specific integrated circuits (ASICs), etc. can also be used either alone or in combinations including analog and digital combinations for the present invention. The present invention can be incorporated into an integrated circuit, be an integrated circuit, etc.

The present invention can also incorporate at an appropriate location or locations one or more thermistors (i.e., either of a negative temperature coefficient [NTC] or a positive temperature coefficient [PTC]) to provide temperature-based load current limiting.

As an example, when the temperature rises at the selected monitoring point(s), the phase dimming of the present invention can be designed and implemented to drop, for example, by a factor of, for example, two. The output power, no matter where the circuit was originally in the dimming cycle, will also drop/decrease by a some factor. Values other than a factor of two (i.e., 50%) can also be used and are easily implemented in the present invention by, for example, changing components of the example circuits described here for the present invention. As an example, a resistor change would allow and result in a different phase/power decrease than a factor of two. The present invention can be made to have a rather instant more digital-like decrease in output power or a more gradual analog-like decrease, including, for example, a linear decrease in output phase or power once, for example, the temperature or other stimulus/signal(s) trigger/activate this thermal or other signal control.

In other embodiments, other temperature sensors may be used or connected to the circuit in other locations. The present invention also supports external dimming by, for example, an external analog and/or digital signal input. One or more of the embodiments discussed above may be used in practice either combined or separately including having and supporting both 0 to 10 V and digital dimming. The present invention can also have very high power factor. The present invention can also be used to support dimming of a number of circuits, drivers, etc. including in parallel configurations. For example, more than one driver can be put together, grouped together with the present invention. Groupings can be done such that, for example, half of the dimmers are forward dimmers and half of the dimmers are reverse dimmers. Again, the present invention allows easy selection between forward and reverse dimming that can be performed manually, automatically, dynamically, algorithmically, can employ smart and intelligent dimming decisions, artificial intelligence, remote control, remote dimming, etc.

The present invention may be used in conjunction with dimming to provide thermal control or other types of control to, for example, a dimming LED driver. For example, embodiments of the present invention may also be adapted to provide overvoltage or overcurrent protection, short circuit protection for, for example, a dimming LED or OLED driver, etc., or to override and cut the phase and power to the dimming LED driver(s) based on any arbitrary external signal(s) and/or stimulus. The present invention can also be used for purposes and applications other than lighting—as an example, electrical heating where a heating element or elements are electrically controlled to, for example, maintain the temperature at a location at a certain value. The present invention can also include circuit breakers including solid state circuit breakers and other devices, circuits, systems, etc. that limit or trip in the event of an overload condition/situation. The present invention can also include, for example analog or digital controls including but not limited to wired (i.e., 0 to 10 V, 0 to 3 V, other voltage ranges, RS 232, RS485, IEEE standards, SPI, I2C, other serial and parallel standards and interfaces, etc.), wireless, powerline, etc. and can be implemented in any part of the circuit for the present invention. The present invention can be used with a buck, a buck-boost, a boost-buck and/or a boost, flyback, or forward-converter design, topology, implementation, etc.

A dimming voltage signal, VDIM, which represents a voltage from, for example but not limited to, a 0-10 V or 0 to 3 V Dimmer can be used with the present invention, when such a VDIM signal is connected, the output as a function time or phase angle (or phase cut) will correspond to the inputted VDIM.

Other embodiments can use comparators, other op amp configurations and circuits, including but not limited to error amplifiers, summing amplifiers, log amplifiers, integrating amplifiers, averaging amplifiers, differentiators and differentiating amplifiers, etc. and/or other digital and analog circuits, microcontrollers, microprocessors, complex logic devices, field programmable gate arrays, etc.

The present invention includes implementations that contain various other control circuits including, but not limited to, linear, square, square-root, power-law, sine, cosine, other trigonometric functions, logarithmic, exponential, cubic, cube root, hyperbolic, etc. in addition to error, difference, summing, integrating, differentiators, etc. type of op amps. In addition, logic, including digital and Boolean logic such as AND, NOT (inverter), OR, Exclusive OR gates, etc., complex logic devices (CLDs), field programmable gate arrays (FPGAs), microcontrollers, microprocessors, application specific integrated circuits (ASICs), etc. can also be used either alone or in combinations including analog and digital combinations for the present invention. The present invention can be incorporated into an integrated circuit, be an integrated circuit, etc.

The present invention, although described for example, but not limited to, for motion and light/photodetection control, can and may also use other types of stimuli, input, detection, feedback, response, etc. including but not limited to sound, vibration, frequencies above and below the typical human hearing range, temperature, humidity, pressure, light including below the visible (i.e., infrared, IR) and above the visible (i.e., ultraviolet, UV), radio frequency signals, combinations of these, etc. For example, the motion sensor may be replaced or augmented with a sound sensor (including broad, narrow, notch, tuned, tank, etc. frequency response sound sensors) and the light sensor could consist of one or more of the following: visible, IR, UV, etc. sensors. In addition, the light sensor(s)/detector(s) can also be replaced or augmented by thermal detector(s)/sensor(s), etc.

The example embodiments disclosed herein illustrate certain features of the present invention and not limiting in any way, form or function of present invention. The present invention is, likewise, not limited in materials choices including semiconductor materials such as, but not limited to, silicon (Si), silicon carbide (SiC), silicon on insulator (SOI), other silicon combination and alloys such as silicon germanium (SiGe), etc., diamond, graphene, gallium nitride (GaN) and GaN-based materials, gallium arsenide (GaAs) and GaAs-based materials, etc. The present invention can include any type of switching elements including, but not limited to, field effect transistors (FETs) of any type such as metal oxide semiconductor field effect transistors (MOSFETs) including either p-channel or n-channel MOSFETs of any type, junction field effect transistors (JFETs) of any type, metal emitter semiconductor field effect transistors, etc. again, either p-channel or n-channel or both, bipolar junction transistors (BJTs) again, either NPN or PNP or both, heterojunction bipolar transistors (HBTs) of any type, high electron mobility transistors (HEMTs) of any type, unijunction transistors of any type, modulation doped field effect transistors (MODFETs) of any type, etc., again, in general, n-channel or p-channel or both, vacuum tubes including diodes, triodes, tetrodes, pentodes, etc. and any other type of switch, etc.

The present invention can, for example, use shorter (i.e., blue) wavelength light to simulate and awake or support waking and healthy state functionality and use longer (i.e., yellow, amber, red, etc.) wavelength light to promote sleep and rest state. For example, use amber OLED(s) for sleep and blue OLED(s) for waking and to simulate the exposure to natural sunlight. The OLEDs can be separate colors, panels, or integrated, layered, etc. colors on the same panel and can be of any type and construction. The present invention can use external information such as time of day/night, light levels, computers, websites, smart phones, clocks, atomic clocks and other wired and wireless timing information, to determine whether to have amber (or yellow or red, etc.), blue or both turned on. AC power, solar power, batteries, or a combinations of these, etc. can be used to provide power to the OLEDs. Embodiments of the present invention can use a portable OLED panel or panels, other types and sizes (from small to very larger and bigger including tiled, stacked, etc. panels) panels including troffers, task lamps, bed lamps, table lamps, under counter, over counter, vanity, wall, ceiling, sconce, luminaries, sleep detectors, wearable sleep detectors and circadian rhythm detectors, etc. Implementations of the present invention can be a fluorescent tube replacement of any length and any diameter that contains multiple color light sources which can be controlled (i.e., turned on, dimmed) in ways to produce shorter visible wavelength containing light for waking up and waking hours and produce longer visible wavelength containing light with the absence of or greatly reduced shorter wavelength content light for sleeping and resting.

The present invention can use edge emitting LED light sources and displays, waveguide LED sources and displays, etc. The present invention can use computer monitors/displays and TVs, smart phones, tablets, iPads, iPhones, iPods, Android devices including, but not limited to, smart cellular phones and tablets, and other color displays, monitors, personal digital assistants, etc. It can use photosensors, motion sensors, proximity sensors, radio frequency identification (RFID), cell phone signals, Bluetooth, WiFi, Wimax, Zigbee, Zwave, other infrared, optical, light, electromagnetic, electromagnetic waves, radio frequency (RF) including, but not limited to the frequency spectrum from less than 1 MHz or kHz to greater than 1 THz or 10 s or 100 s of THz, etc., to smart phones, tablets, global positioning systems (GPS), voice activated, voice recognition, sound activation, selective sound activation, temperature activation, humidity action, motion activation, infrared activation, etc. combinations of these, etc.

The present invention can be synchronized, set, programmed, etc. to work with internal and external stimuli, signals and input to provide lighting to improve health care, function, operation, well being and also provide other features including warnings, alerts, alarms, etc. that can be, for example, but not limited to, audible, vision, audio, visual, lighting, sound, light, e-mail, text messages, phone calls, web content, web alerts, etc.

Embodiments of the present invention can be battery powered and charged by any method including AC battery chargers, AC/DC battery chargers, inverters, converters, solar energy, mechanical energy, energy harvesting or one or more types, combinations of these, car/automobile chargers, etc.

The present invention can also be used to provide relatively small illumination at night of appropriate wavelengths and can be integrated into a single light source and sensor unit to provide lighting sufficient for sleeptime/nighttime use and egress for, for example, children and adults including more aged and senior adults and parental or other (including, but not limited to nursing, nurse assistant, care giver, hospital, rest home, hospice, trauma, emergency room and similar environments, recovery, rehabilitation, assisted living, elderly living, senior care, etc. centers/facilities, etc.). The present invention's lighting can be used for, for example but not limited to, seniors, families, businesses, residences, homes, houses, elderly, physically impaired people and persons, etc. to signal, alarm and/or alert others of an emergency, an intrusion, a fire, a fall, an injury, toxic or explosive gases, loss of heating, water leakage, etc., by for example flashing lights, on-off lights at certain periods of repetition, different colors flashing, different patterns of colors, different intensities and dimming, etc., combinations of these, etc. In some cases, the interior/indoor lights can be set to full on/full brightness while the exterior/outdoor lights can be set to flashing or other modes including but not limited to those discussed herein.

The present invention can be made into light sources, including but not limited to sheet light sources, which can incorporate solar cells either on the front or the back, and optional energy storage such as batteries to create a light source that can be powered when there is no sunlight or can also act as a privacy screen and/or temperature reducer over windows by absorbing and blocking the sunlight (and potentially associated heat and UV rays) from entering the space on the interior side of the window while still powering and providing energy to the light sources to illuminate the interior space(s).

An example of the present invention includes, but is not limited to, a light source for train, bus, airplane, ship, boat, yacht, recreational vehicle (RV), SUV, limousine, van, submersible vehicles including, but not limited to, submarines, Navy boats, commercial jets, etc.

The present invention can be used to produce various effects in, for example, a long distance travel by train, boat or plane in which the users can chose from soothing or exciting colors, certain wavelengths of light to help induce, reset, etc. circadian rhythms and melatonin production or suppression, etc.

The present invention can be used, for example, on a commercial airplane to allow the passenger to adjust the local lighting by using, for example, Bluetooth, WiFi, or any other wireless method, way, protocol, etc. to, for example, communicate with the light/lamp to dim, change color temperature, change color, turn off, turn on, tilt, and/or combinations of these, etc.

The present invention can be made to be transparent or nearly transparent and mounted on, embedded in, attached to, etc. windows to control, monitor and permit appropriate wavelength light transmission.

Embodiments of the present invention can provide emulation/simulation/etc. of the Sun's (e.g., solar) spectrum of light including from pre-dawn to post-dusk and can optionally augment/supplement/synchronize/set the spectrum especially the full visible spectrum.

Embodiments of the present invention can, among other things, track the Sun or time/phase shift the Sun's spectrum and provide such spectrum lighting at any time including offset times for, for example, shift workers. Embodiments of the present invention can provide exact replicas of the Sun's spectrum or add or subtract from the Sun's spectrum.

Embodiments of the present invention can provide exact timing to match the local time or other time zones so as to train and entrain, sync, etc. a person's or persons' circadian rhythm to a local or other time zone for work, travel, vacation, etc. uses and purposes. The present invention can take and gather information from numerous sources including but not limited to the web/internet, the local and global environment, and the user, etc. and, for example, precisely or approximately put out a spectrum that emulates that of the Sun or deviates from that of the Sun at certain times and moments and locations which can be programmed by the user or by others to, for example, account for cloudy days, rainy days, and other weather and Sun/solar related, etc. conditions, matters, issues, considerations, etc. The present invention can adjust, adapt, modify, etc., and be programmed to respond to various human factors and environmental conditions.

The present invention can also be used to provide a smart, intelligent and interactive light source to treat seasonal affective disorder (SAD) among other light/phototherapy treatments/applications/needs/etc. including receiving signals from one or more sensors and detectors including, but not limited to wired and wireless signals, feedback, information, etc. from one or more devices including with wearable devices and other sensors that can detect, for example, but not limited to, heart rate, blood pressure, phase of the circadian rhythm cycle, other information about circadian rhythm, ambient light, pressure, movement, electroencephalogram/electroencephalography (EEG), electrocardiography/electrocardiogram (EKG or ECG), brain waves, oxygen level, brain waves, muscle movement, body temperature, pulse rate, actimetry, sleep actigraphs, temperature, polysomnography (PSG), mood, emotional state, etc. Wearable devices can include, but are not limited to, wrist devices, or watch-shaped devices worn on the wrist of the non-dominant arm, detectors and sensors, sleep management and monitoring sensors, systems, etc. including for awake, REM, deep sleep, various other states of sleep and wake, etc., delayed sleep phase disorder, perspiration, orientation, location, vertical or horizontal sensing, etc., speech, speech patterns, voice, weather, etc.

The present invention allows for scheduling/programming of events remotely including for persons who are unable to do so themselves which can also include remote scheduling, programming, monitoring, control, etc. The present invention can also be used to treat and/or assist in the treatment of dementia and related conditions. The present invention can also provide power for other uses, functions including but not limited to fans, motors, heaters, blowers, fan blades, security cameras, surveillance cameras, monitors, monitoring systems, web-based cameras, motorized cameras, etc., USB and other charging, auxiliary power, etc., battery backup, emergency batteries, microphones, speakers, sensors, WiFi, wireless power, combinations of these, etc. In some embodiments of the present invention, various wireless approaches can be used that for example, but are not limited to, involve WiFi and Bluetooth to communicate with devices including but not limited to smart phones, iPods, iPads, tablets, computers, laptops, etc. along with direct communication including, but not limited to, wireless remote controls, voice control, voice recognition, etc. via Bluetooth, ISM, other wireless frequencies, etc. For example, a microphone that can communicate via Bluetooth and/or ISM or other wireless frequencies can be used to communicate with the present invention. In some embodiments of the present invention, a buck, buck-boost, boost-buck, and/or boost switching topology is used to provide power for the present invention.

As an example, a buck circuit can be used to provide AC to DC regulated power to the present invention. An example of an efficient way of providing such power is to for example have the buck circuit be controlled based on the lowest and strictest required regulation voltage that typically is used for the control circuits such as, for example, the integrated circuits which could, for example, consist of but is not limited to a microcontroller, microprocessor, FPGA, DSP, CLD, etc., one or more of these or each of these, wireless or wired ICs, interfaces, devices, protocols, etc. including but not limited to, WiFi, Bluetooth, IEEE 801, ISM frequencies, other bands and frequencies, I2C, RS232, RS485, DMX, DALI, SPI, USB, serial, etc., combinations of these including one or more of the same or different ones, etc. that is used with one or more windings (as discussed in U.S. patent application Ser. No. 13/674,072, filed Jun. 2, 2013 for a “Dimmable LED Driver with Multiple Power Sources” which is incorporated herein by reference for all purposes) on the buck inductor to provide multiple outputs including, for example, but not limited to, typically 3 V to 5 V for the control electronics, 5 V to 15 V to 20 V for the power devices including the gate drive for the power transistors including FETs and in some embodiments bipolar junction transistors (BJTs) and Darlingtons and IGBTs. In addition to these windings, a winding or windings for, for example, can also be used to provide power to the LEDs and/or OLEDs as well as power for other needs and applications including fans, motors, USB, battery chargers, etc. Linear regulation, linear regulators, switching regulators, voltage regulators, current regulation, current regulators, shunt, regulation, shunt regulators, combinations of these, etc. may be used.

The present invention can be used to aid in circadian rhythm regulation and cycle synchronization. The present invention can aid in correcting sleep disorders. The present invention can use input, feedback, etc. including human physiological and biological input and feedback and environmental (including, but not limited to, temperature, time of day or night, ambient light, light spectrum, etc.) to control and monitor the light including the colors/wavelengths of the light, etc.

The present invention can be used for personal or professional use and applications. The present invention can be, for example, but not limited to being used in hospitals, rest homes, senior care homes, rehabilitation facilities, short term and long term care facilities, homes, residences, commercial and industrial buildings and locations, in confined spaces, in spaces devoid of natural light, on ships, buses, boats, planes, aircraft, submarines, vessels, all times of marine, ground, air and space vehicles including transport and working environments, spaces, vehicles, etc.

The present invention can use actimetry, sleep actigraphs which can be of any form including watch-shaped and worn on the wrist of the non-dominant arm, temperature, EEG, wrist, body movements, polysomnography (PSG) and other such techniques, etc.

The present device can be made into light sources, including but not limited to sheet light sources, which can incorporate solar cells either on the front or the back, and optional energy storage such as batteries to create a light source that can be powered when there is no sunlight or can also act as a privacy screen and/or temperature reducer over windows by absorbing and blocking the sunlight (and potentially associated heat and UV rays) from entering the space on the interior side of the window while still powering and providing energy to the light sources to illuminate the interior space(s).

Ballasts can be used as power sources and supplies with multiple uses, applications, voltages, power, current and voltage control, etc.

The present invention can be used to provide, control, dimming, on/off, monitoring, logging, decision making, etc. of providing power including wall power including in a single or dual wall plug or higher count in a single gang, two gang, multiple gang box size or as a plug-in extender, etc. The present invention can be wired, wireless, etc. The present invention can be mounted/installed in, for example but not limited to, in a standard wall outlet box, a wall dimmer, an on/off switch, a light socket, including but not limited to an A-lamp socket, a E26 socket, etc.

The present invention can monitor, store, log, etc., electrical parameters including, but not limited to, current, voltage, power, power factor, apparent power, real power, AC current, DC current, AC voltage, DC voltage, etc. The present invention can select between dimming, dimming with on/off and on/off only by automatic, manual including switch(es), remote control, detection and analysis, etc. The present invention can, for example, measure the AC input voltage and produce a scaled version of the AC input voltage, measure the AC input current and produce a scaled version of the AC input current, measure any DC offsets to the input current, voltage, power, etc. measure the output current, voltage, power, etc. One embodiment for measuring the AC input voltage involves the use of high resistance resistors and one or more op amps. Such embodiments can involve level shifting if needed. Measuring either the input current or voltage or both can be accomplished by the use of op amps; for example, the current can be measured by measuring the voltage across a relatively low value resistance and then applied, and voltage shifted if needed, using an op amp or op amps. In some embodiments of the present invention, various wireless approaches can be used that for example, but are not limited to, involve WiFi and Bluetooth such that devices including but not limited to smart phones, iPods, iPads, tablets, computers, laptops, etc. along with direct communication including, but not limited to, wireless remote controls, voice control, voice recognition, etc. via Bluetooth, ISM, other wireless frequencies, etc. For example, a microphone that can communicate via Bluetooth and/or ISM or other wireless frequencies can be used to communicate with the present invention.

The present invention can be used to provide assisted care or monitoring in general including using voice commands, voice recognition, wearable device(s) information, wired and wireless panic buttons, proximity sensors, motion sensors, sound sensors, etc. The present invention can take, use, analyze, make decisions, etc. based on data, signals, information, etc., from one or more sensors and detectors including, but not limited to wired and wireless signals, feedback, information, etc. from one or more devices including with wearable devices and other sensors that can detect, for example, but not limited to, heart rate, blood pressure, phase of the circadian rhythm cycle, EEG, EKG, oxygen level, brain waves, muscle movement, body temperature, pulse rate, mood, emotional state, location, GPS, elevation, sound, mechanical, movement, time duration, vibration, sound, pressure, accelerometer(s), sound spectrum, ultrasound, sonar, etc.

Such signals, input, feedback, information, etc. can be used to, for example, to set the level, spectrum and intensity, emulated sunlight spectrum, white temperature, color temperature, lighting sensors, duration and intensity of treatment, etc. In addition, sensors can include light sensors, photosensors, spectrum analyzers including optical spectrum analyzers, light sensors with notch filters, motion sensors, proximity sensors, radio frequency identification (RFID), cell phones, smart phones, tablets, etc. Smart phones, tablets, laptops, computers, dedicated control and/or interface units, etc. may be used to, for example, but not limited to, transmit and/or process the information via applications or apps or can use apps to display, store, log, analyze, etc. data, results, performance, control, provide feedback, etc. The present invention can incorporate and use open platforms including but not limited to Google Fit, Apple HealthKit, etc.

Telephone-based, Web-based, Cloud-based, etc., Cell phone based, combinations of these, etc. can be used to transmit, receive, communicate, recognize, alert, warn, contact, control, monitor, etc. In some embodiments of the present invention, various wireless approaches can be used that for example, but are not limited to, involve WiFi and Bluetooth such that devices including but not limited to smart phones, iPods, iPads, tablets, computers, laptops, etc. along with direct communication including, but not limited to, wireless remote controls, voice control, voice recognition, etc. via Bluetooth, ISM, other wireless frequencies, etc. For example, a microphone that can communicate via Bluetooth and/or ISM or other wireless frequencies can be used to communicate with the present invention. The present invention can take a number of actions including flashing lights, contacting specified people, agencies, groups, services, departments, entities, individuals, etc. via web, mobile, smart, etc., cellular phones, tablets, other mobile devices, etc., land line, conventional phones, e-mails, text messages, cellular services, etc. In embodiments of the present invention, the absence of a signal, information, and/or response including but not limited to physiological including but not limited to blood pressure, heart rate, oxygen levels, insulin levels, temperature, other physiological monitors, sensors, etc., motion, proximity, temperature, humidity, room occupancy, room temperature, electrical power usage, lack of electrical power usage, water flow, water usage, gas usage, carbon monoxide and other gas sensing, lights and other appliances turned off or turned on (state of usage, time of usage, duration of usage), voice recognition, voice commands, sounds, movements, breakage, noise(s), patterns, etc.

The present invention can be used to aid in circadian rhythm regulation and cycle synchronization. The present invention can aid in correcting sleep disorders. The present invention can use input, feedback, etc. including human physiological and biological input and feedback and environmental (including, but not limited to, temperature, time of day or night, ambient light, light spectrum, etc.) to control and monitor the light including the colors/wavelengths of the light, etc.

The present invention can be used for personal or professional use and applications. The present invention can be, for example, but not limited to being used in hospitals, rest homes, senior care homes, rehabilitation facilities, short term and long term care facilities, homes, residences, commercial and industrial buildings and locations, in confined spaces, in spaces devoid of natural light, on ships, buses, boats, planes, aircraft, submarines, vessels, all times of marine, ground, air and space vehicles including transport and working environments, spaces, vehicles, etc.

Embodiments of the present invention can be made into light sources, including but not limited to sheet light sources, which can incorporate solar cells either on the front or the back, and optional energy storage such as batteries to create a light source that can be powered when there is no sunlight or can also act as a privacy screen and/or temperature reducer over windows by absorbing and blocking the sunlight (and potentially associated heat and UV rays) from entering the space on the interior side of the window while still powering and providing energy to the light sources to illuminate the interior space(s).

The circadian rhythm alignment lighting system disclosed herein provides a variety of light sources and light controls that can be used to improve circadian rhythms, thereby greatly improving health, sleep, work, concentration, focus, performance, etc.

While detailed descriptions of one or more embodiments of the invention have been given above, various alternatives, modifications, and equivalents will be apparent to those skilled in the art without varying from the spirit of the invention. Therefore, the above description should not be taken as limiting the scope of the invention, which is defined by the appended claims.

What is claimed is:

1. A lighting system comprising:
a plurality of light sources with multiple light colors configured to affect circadian rhythms; and
a power supply configured to power the plurality of light sources and to control an overall output color.

2. The lighting system of claim 1, wherein the plurality of light sources and the power supply are embodied in a fluorescent lamp replacement.

3. The lighting system of claim 2, wherein the power supply comprises an AC input and a ballast input configured to receive power from a fluorescent lamp ballast.

4. The lighting system of claim 3, wherein the power supply is configured to automatically select between providing power from the AC input or from the ballast input depending at least in part on whether the fluorescent lamp ballast is present in a fixture.

5. The lighting system of claim 3, wherein the power supply comprises a switch configured to manually select between providing power from the AC input or from the ballast input depending at least in part on whether the fluorescent lamp ballast is present in a fixture.

6. The lighting system of claim 3, wherein the power supply comprises a heater simulation circuit configured to simulate a fluorescent lamp heater circuit for the fluorescent lamp ballast.

7. The lighting system of claim 3, wherein the power supply comprises an overvoltage protection circuit connected to the ballast input.

8. The lighting system of claim 2, further comprising at least one sensor configured to monitor when a person is looking at the plurality of light sources.

9. The lighting system of claim 2, wherein the plurality of light sources comprises at least one blue OLED panel and at least one amber OLED panel.

10. The lighting system of claim 9, wherein the power supply is configured to control an output of the at least one blue OLED panel and the at least one amber OLED panels simultaneously to produce a white light output.

11. The lighting system of claim 1, further comprising a controller configured to control the overall output color based at least in part on time of day.

12. The lighting system of claim 1, further comprising a controller configured to turn on the plurality of light sources to wake a person as a light alarm.

13. The lighting system of claim 1, further comprising a microphone and a controller configured to control the plurality of light sources based at least in part on sound detected by the microphone.

14. The lighting system of claim 13, wherein the controller is configured to provide an indication of sound levels over a threshold by controlling the plurality of light sources.

15. The lighting system of claim 1, wherein the plurality of light sources comprise a combination of OLED and LED light sources.

16. The lighting system of claim 1, wherein the plurality of light sources comprise OLED panels placed in multi-sided arrangement.

17. The lighting system of claim 16, wherein each side of the multi-sided OLED panels is configured to output light of a different color selected to affect circadian rhythms differently.

18. The lighting system of claim 2, wherein the power supply is configured to receive power from multiple ballast outputs.

19. The lighting system of claim 18, wherein different power inputs to the power supply are isolated.

20. The lighting system of claim 18, wherein different power inputs to the power supply are non-isolated.
